(19) United States
(12) Patent Application Publication
(10) Pub. No.: US 2011/0223283 A1
(43) Pub. Date: Sep. 15, 2011

(54) TRANSFER METHOD

(76) Inventors: Karsten Mathias Kragh, Viby J (DK); Rie Mejldal, Ostbirk (DK); Rene Mikkeisen, Brahand (DK)

(21) Appl. No.: 13/055,259

(22) PCT Filed: Jul. 16, 2009
(86) PCT No.: PCT/IB2009/006526

§ 371 Date: May 13, 2011

Related U.S. Application Data
(60) Provisional application No. 61/083,306, filed on Jul. 24, 2008.

(30) Foreign Application Priority Data
Jul. 24, 2008 (GB) 0813581.6

(51) Int. Cl. C12P 19/44; C12P 19/18; A21D 10/02; A21D 8/04

(52) U.S. Cl. 426/10; 435/97; 426/64

(57) ABSTRACT

A method of glycosylating a lipid having a free hydroxyI group, the method comprising contacting said lipid with a source of monosaccharide moiety and a transglycosidase enzyme, is disclosed. In particular, a method for in situ production of the glycosylated lipid in a food product is disclosed.

### FIELD OF THE INVENTION

This invention relates to novel uses of an enzyme. In particular, the invention relates to use of a transglycosidase enzyme in a number of novel applications, especially glucose transfer to lipids and food products having improved emulsification properties.

### DESCRIPTION OF THE PRIOR ART

A transglucosidase enzyme (E.C. 3.2.1.20) (also known as D-glucosyltransferase or α-glucosidase) is commercially available as TGL-500™ from Danisco/Genencor. The enzyme may be derived from fungal sources, in particular Aspergillus niger (Genbank acc nr D45356.1 and SwissProt accession no. P56526.1).

The primary application of the above transglucosidase is in the production of isomalto-oligosaccharides (IMO) which are small α-1,6 branched oligosaccharides—see ‘Novel α-Glucosidase from Aspergillus nidulans with Strong Transglycosylation Activity’ N. (Kato et al. (2002)). Commercially available IMO products are produced from maltose/maltodextrin by applying transglucosidase: the final product contains a mixture of isomaltose, isomaltotriose and panose. TGL-500™ catalyzes both hydrolytic and transfer reactions on incubation with α-D-gluco-oligosaccharides. Transfer occurs most frequently to the hydroxyl group at C-6 producing isomaltose from D-glucose and panose from maltose.

A one step transglucosidase catalyzed synthesis of alpha-alkylglucosides has been shown previously (Busquet, M.-P. et al. (1998). Monsan, P. et al.). Transglucosidases from Talaromyces duponti and Aspergillus niger have been used to catalyze the transfer of glucosyl units from alpha-1,4 linked carbohydrate donors to alkyl alcohols such as 1-butanol generating alkyl-glucosides.

Glycolipids and phospholipids dominate the endogenous polar lipid fraction of wheat flour. Glycolipids, in particular, act as surfactants and/or emulsifiers during bread making. Glycolipids positively affect the volume, texture and staling of bread (Pomeranz, Y. (1971)). Therefore glycolipids are useful as emulsifiers and/or surfactants during bread making to improve the properties such as volume, texture and rate of staling of bread. Glycolipids in general improve emulsification properties of bakery products.

### SUMMARY OF THE INVENTION

The invention comprises in a first aspect method of glycosylating a lipid having a free hydroxyl (—OH) group, the method comprising contacting said lipid with a source of monosaccharide moiety, as defined herein, and a transglycosidase enzyme, as defined herein.

The invention comprises in a second aspect a method of transfer of a monosaccharide moiety to a lipid having a free hydroxyl (—OH) group, by contacting the lipid with a source of monosaccharide moiety, as defined herein, and a transglycosidase enzyme, as defined herein.

The invention comprises in a third aspect a method of improving the emulsification properties of a food product, the method comprising contacting the food product or food product intermediate, as defined herein, with a source of monosaccharide moiety, as defined herein, a lipid, as defined herein, and a transglycosidase enzyme, as defined herein.

The invention comprises in a fourth aspect a method for in situ production of a glycosylated lipid in a food product, the method comprising contacting the food product or food product intermediate as defined herein, with a source of monosaccharide moiety, as defined herein, a lipid, as defined herein, and a transglycosidase enzyme, as defined herein.

The invention comprises in a fifth aspect use of a transglycosidase enzyme, as defined herein, for the transfer of a monosaccharide moiety to a lipid, as defined herein, having a free hydroxyl (—OH) group.

The invention comprises in a sixth aspect use of a transglycosidase enzyme, as defined herein, for improving the emulsification properties of a food product, said food product including a lipid, as defined herein, having a free hydroxyl (—OH) group, and a source of monosaccharide moiety, as defined herein.

The invention comprises in further aspects a food product improving composition and food products formed by the above methods. These are described in more detail hereinafter.

### BRIEF DESCRIPTION OF THE DRAWINGS

FIG. 1 illustrates the amounts of monoglucosyl monoglyceride (MGMG) and diglucosyl monoglyceride (DGMG) generated in vitro by TGL-500 in Example 1;

FIG. 2 illustrates the amounts of monoglucosyl diglyceride (MGDG) generated in vitro by TGL-500 in Example 1;

FIG. 3 illustrates the mono-galactosyl/glucosyl monoglyceride (MGMG) content (%) in the dough of Example 2 after proof;

FIG. 4 illustrates the di-galactosyl/glucosyl monoglyceride (DGMG) content (%) in the dough of Example 2 after proof;

FIG. 5 illustrates the mono-galactosyl/glucosyl diglyceride (MGDG) content (%) in the dough of Example 2 after proof;

FIG. 6 illustrates the di-galactosyl/glucosyl diglyceride (DGDG) content (%) in the dough of Example 2 after proof;

FIG. 7 illustrates the mono-galactosyl/glucosyl monoglyceride (MGMG) content (%) in the bread crumb of Example 2;

FIG. 8 illustrates the di-galactosyl/glucosyl monoglyceride (DGMG) content (%) in the bread crumb of Example 2;

FIG. 9 illustrates the mono-galactosyl/glucosyl diglyceride (MGDG) content (%) in the bread crumb of Example 2;

FIG. 10 illustrates the di-galactosyl/glucosyl diglyceride (DGDG) content (%) in the bread crumb of Example 2;

FIG. 11 illustrates the in vitro generation of MGMG and DGMG by TGL-500 applying maltodextrin as a substrate in Example 3; and

FIG. 12 illustrates the in vitro generation of MGMG and DGMG by TGL-500 applying sucrose as a substrate.

### DETAILED DESCRIPTION OF PREFERRED EMBODIMENTS

Preferred embodiments of the invention are described below. Unless specifically stated otherwise, the preferred embodiments apply to all aspects of the invention, including all methods, uses and products described and claimed herein.

### Glycosylation/Transglycosidase

The present invention comprises in one aspect a method of glycosylating a lipid, the method comprising contacting said lipid with a source of monosaccharide moiety, as defined herein, and a transglycosidase enzyme.

In this specification the terms ‘glycosylation’ and ‘glycosylating’ mean the formation of a glycoside bond between the hemiacetal hydroxyl group at the anomeric position of the monosaccharide moiety and the oxygen of the free hydroxyl group on the lipid acceptor molecule, with the consequent elimination of a water molecule. The glycosidic bond formed may be an α- or β-glycoside bond.

The present invention is based on the surprising finding that a transglycosidase enzyme, as defined herein, can transfer a monosaccharide moiety, in particular a glucose moiety, to a lipid, as defined herein. Use of lipids, in particular mono- and diglycerides, as acceptor substrates for a glucose moiety in a dough system, generates glucoglycerolipids in situ: such compounds are expected to exhibit strong emulsification properties in dough systems. It is known (see Carter et al. (1956), Carter et al. (1961) and Carter et al. (1961), as well as Pomeranz, Y. (1987)) that wheat flour contains very small amounts of galactosyl modified mono- and di-glycerides, such as mono- and di-galactosyl monoglycerides and that these have very good emulsifying effects.

In this specification the term ‘transglycosidase’ is intended to cover any enzyme capable of transferring a monosaccharide moiety, as defined below, from one molecule to another. The term ‘transglucosidase’ is used when the monosaccharide moiety is a glucose moiety. Preferably, the transglycosidase enzyme is a transglucosidase enzyme.

As outlined above, it is known that transglycosidases (in particular, transglucosidases) can transfer monosaccharide moieties to other carbohydrates: however, it has not previously been shown that an enzyme in this class can transfer a monosaccharide moiety to a lipid.

In one embodiment, the transglycosidase is classified in enzyme classification (E.C.) 3.2.1.20.

In one embodiment, the transglycosidase enzyme is classified in Glycoside Hydrolase Family 31 of the Carbohydrate-Active Enzymes (CAZy) database. This database is described at http://www.cazy.org/ and in Coutinho, P. M. & Henrissat, B. (1999). This classification system is based on structural and sequence features rather than substrate specificity: as there is a direct relationship between sequence and folding similarities; such a classification: (i) reflects the structural features of these enzymes better than their sole substrate specificity, (ii) helps to reveal the evolutionary relationships between these enzymes, and (iii) provides a convenient tool to derive mechanistic information. In this classification, glycoside hydrolases (EC 3.2.1.-) are a widespread group of enzymes which hydrolyse the glycosidic bond between two or more carbohydrates or between a carbohydrate and a non-carbohydrate moiety. Further description of the classification of glycoside hydrolases (glycosidases and transglydosidases can be found in Henrissat B (1991), Henrissat B, Bairoch A (1993), Henrissat B, Bairoch A (1996), Davies G, Henrissat B (1995) and Henrissat B, Davies G J (1997)).

In another embodiment transglycosidases or transglucosidases can be described functionally in terms of the reaction that they perform. Enzymes that can transfer sugar moieties to a lipid under the conditions described in Example 1 are useful in the present invention.

Enzymes that are suitable for use in the present invention can be identified by their ability to transfer sugar moieties from an appropriate monosaccharide source to a C10 monoglyceride in vitro.

An example of an assay that can be used to test a candidate enzyme for use in the present invention to identify whether it can transfer a sugar moiety to a lipid comprises the steps of:
incubating a candidate enzyme with a solution of C10 monoglyceride and an apporopriate monosaccharide source; and
identifying whether a sugar has been transfered to the monoglyceride by Liquid Chromatography-mass spectrometry analysis.

Such an assay may be performed, for example, under the conditions described in Example 1 with TGL-500 replaced with the candidate enzyme.

In one embodiment, the transglycosidase enzyme is obtainable or is obtained from a living organism. Suitable transglycosidase enzymes are of bacterial or fungal origin. Preferred are transglycosidase enzymes of fungal origin.

In one embodiment, the transglycosidase enzyme is of fungal origin or has at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity with a transglucosidase enzyme of fungal origin.

In a preferred embodiment, the transglycosidase enzyme originates from an Aspergillus species, especially an Aspergillus species selected from the group consisting of Aspergillus niger, Aspergillus awamori, Aspergillus terreus, Aspergillus oryzae, Aspergillus nidulans, Aspergullus fumigatus and Aspergillus clavatus or has at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity with a transglucosidase enzyme originating from an Aspergillus species.

In a particularly preferred embodiment, the transglycosidase enzyme is Aspergillus niger transglucosidase encoded by SEQ ID No 1 or a sequence having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity therewith.

The transglucosidase enzyme may be post-translationally modified, for example by cleavage of a signal sequence or by glycosylation.

### Amino Acid Sequences

Amino acid sequences of transglycosidase enzymes capable of transferring a monosaccharide moiety to a lipid, as defined herein, particularly transglycosidase enzymes having the amino acid sequence of SEQ ID No. 2 defined below, may be used in the present invention.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The protein used in the present invention may be used in conjunction with other proteins, particularly other enzymes, for example amylases, proteases or lipases. Thus the present invention also covers a composition comprising a combination of enzymes wherein the combination comprises the transglycosidase enzyme used in the present invention and another enzyme, which may be, for example, another transglycosidase enzyme as described herein. This aspect is discussed in a later section.

### Sequence Identity/Sequence Homology/Variants/Homologues/Derivatives

The present invention also encompasses the use of polypeptides having a degree of sequence identity or sequence homology with amino acid sequence(s) defined herein or with a polypeptide having the specific properties defined herein. The present invention encompasses, in particular, peptides having a degree of sequence identity with SEQ ID No. 2, or SEQ ID No. 4 defined below, or homologues thereof. Here, the term “homologue” means an entity having sequence identity with the subject amino acid sequences or the subject nucleotide sequences. Here, the term “homology” can be equated with “sequence identity”.

The homologous amino acid sequence and/or nucleotide sequence should provide and/or encode a polypeptide which retains the functional activity and/or enhances the activity of the transglycosidase enzyme.

In the present context, a homologous sequence is taken to include an amino acid sequence which may be at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In a particularly preferred embodiment, the transglycosidase enzyme is Aspergillus niger transglucosidase having the sequence shown in SEQ ID No. 2 or SEQ ID No. 4 or a sequence having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity therewith.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools are available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/ and which was firstly described in Altschul et al. (1990) J. Mol. Biol. 215; 403-410.

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools-sequence analysis-ClustalW2. Currently, the exact address of the ClustalW2 tool is www.ebi.ac.uk/Tools/clustalw2.

Thus, the present invention also encompasses the use of variants, homologues and derivatives of any amino acid sequence of a protein as defined herein, particularly those of SEQ ID No. 2 or SEQ ID No. 4 or those encoded by SEQ ID No. 1, defined below.

The sequences, particularly SEQ ID No. 2 or SEQ ID No. 4, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue, with an alternative residue) that may occur i.e. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxylamino acids (Serine, Threonine), large amino acids (Phenylalanine and Tryptophan) and small amino acids (Glycine, Alanine).

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-∈-amino butyric acid*, L-γ-amino butyric acid*, L-α-amino isobutyric acid*, L-∈-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-conservative substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al. (1992), Horwell D C. (1995).

In a preferred embodiment, the transglucosidase enzyme is Aspergillus niger transglucosidase having the amino acid sequence shown in SEQ ID No 2. or SEQ ID No. 4 encoded by a nucleic acid having the sequence shown in SEQ ID No. 1 or an enzyme having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99% amino acid sequence identity therewith.

In one aspect, preferably the sequence used in the present invention is in a purified form. The term “purified” means that a given component is present at a high level. The component is desirably the predominant active component present in a composition.

### Amount/Concentration

The amount of transglycosidase required in the glycosylation method of the present invention is not particularly limited.

In one embodiment, the glycosylation methods of the present invention require an effective amount of the transglycosidase enzyme. In particular, in one embodiment the present invention provides a method of glycosylating a lipid having a free hydroxyl group, the method comprising contacting said lipid with a source of monosaccharide moiety and an effective amount of a transglycosidase enzyme.

In one embodiment the food products of the present invention comprise an effective amount of a transglycosidase enzyme. In particular, in one embodiment the present invention provides a food product improving composition including: a transglycosidase enzyme, as defined herein; a source of monosaccharide moiety, as defined herein; and a lipid, as defined herein.

In this specification the term ‘effective amount’ means an amount of transglycosidase enzyme capable of causing a measurable quantity of monosaccharide moiety to be transferred to the lipid acceptor molecule.

The amount of monosaccharide moiety transferred to the lipid acceptor molecule may be measured using Liquid Chromatography-Mass Spectrometry (LC-MS).

For example, the reduction in the amount of monosaccharide source or the increase in the amount of glycolipid in the reaction mixture may be measured at different time points during the reaction.

The transglycosidase enzyme may be present in any concentration to enable it to perform the above required function of transferring a monosaccharide moiety, in particular a glucose moiety, to a lipid.

In one embodiment, the transglycosidase is present in a concentration of 0.02-200 units of transglucosidase activity (U), preferably 0.08-50 U and most preferably 0.2-20 U per gram of the lipid acceptor.

In one embodiment, the transglycosidase is present in a concentration of 0.0001-1 units of transglucosidase activity (U), preferably 0.0005-0.2 U and most preferably 0.001-0.1 U per mole of the lipid acceptor.

Herein one unit of transglucosidase activity (U) is defined as the amount of enzyme required to produce one micromole of panose per minute when the substrate is maltose.

### Monosaccharide Transferred

The nature of the monosaccharide moiety transferred to the lipid by the transglycosidase enzyme is not particularly critical. The monosaccharide moiety may have the D- or L-configuration. Furthermore, the monosaccharide moiety may be an aldose or ketose moiety.

Suitably, the monosaccharide moiety may have 3 to 8, preferably 4 to 6, more preferably 5 or 6, carbon atoms. In one embodiment, the monosaccharide moiety is a hexose moiety (ie it has 6 carbon atoms), examples of which include aldohexoses such as glucose, galactose, allose, altrose, mannose, gulose, idose and talose and ketohexoses such as fructose and sorbose. Preferably, the hexose moiety is a glucose moiety.

In another embodiment, the monosaccharide moiety is a pentose moiety (ie it has 5 carbon atoms), such as ribose, arabinose, xylose or lyxose. Preferably, the pentose moiety is an arabinose or xylose moiety.

### Monosaccharide Source

The source of monosaccharide moiety to be transferred according to the present invention is not especially critical, provided that it contains a monosaccharide moiety attached to the remainder of the source molecule via a glycosidic bond, which is hydrolysed by the enzyme during the course of the transfer.

However, it is preferred in the present invention that the source of monosaccharide is a higher saccharide (ie a di-, oligo- or polysaccharide) comprising more than one monosaccharide moiety joined together by glycoside bonds, the enzyme acting to hydrolyse one or more glycoside bonds in the higher saccharide and transfer the monosaccharide to the lipid. In this regard, the monosaccharide moieties which form the higher saccharide may be the same or different, and may each independently have the D- or L-configuration.

The monosaccharide moieties which form the higher saccharide may each independently be aldose or ketose moieties, and may have the same or different numbers of carbon atoms. Suitably, each monosaccharide moiety may have 3 to 8, preferably 4 to 6, and more preferably 5 or 6, carbon atoms.

In one embodiment, the monosaccharide moieties which form the higher saccharide are hexose moieties, examples of which include aldohexoses such as glucose, galactose, allose, altrose, mannose, gulose, idose and talose and ketohexoses such as fructose and sorbose. Preferably, the hexose moieties of such a higher saccharide include one or more glucose moieties. In one particularly preferred embodiment, all of the hexose moieties of such a higher saccharide are glucose moieties.

In another embodiment, the monosaccharide moieties which form the higher saccharide are pentose moieties such as ribose, arabinose, xylose or lyxose. Preferably, the pentose moieties of such a higher saccharide are arabinose or xylose moieties.

The monosaccharide moieties which form the higher saccharide are joined together by glycoside bonds. When the monosaccharide moieties are hexose moieties, the glycoside bonds may be 1-α, 1′-α glycoside bonds, 1,2′-glycoside bonds (which may be 1-α-2′ or 1′-β-2′ glycoside bonds), 1,3′-glycoside bonds (which may be 1-α-3′ or 1-β-3′-glycoside bonds), 1,4′-glycoside bonds (which may be 1-α-4′ or 1-β-4′-glycoside bonds), 1,6′-glycoside bonds (which may be 1-α-6′ or 1-β-6′-glycoside bonds), or any combination thereof.

In one embodiment, the higher saccharide comprises 2 monosaccharide units (ie is a disaccharide). Examples of suitable disaccharides include maltose, isomaltose, isomaltulose, lactose, sucrose, cellobiose, nigerose, kojibiose, trehalose and trehalulose.

In another embodiment, the higher saccharide comprises 3 to 10 monosaccharide units (ie is an oligosaccharide) in a chain, which may be branched or unbranched. Preferably, the oligosaccharide comprises 3 to 8, more preferably 3 to 6, monosaccharide units. Examples of suitable oligosaccharides include maltodextrin, maltotriose, maltotetraose, maltopentaose, maltohexaose, maltoheptaose, melezitose, cellotriose, cellotetraose, cellopentaose, cellohexaose and celloheptaose.

In another embodiment, the higher saccharide is a polysaccharide, comprising at least 10 monosaccharide units joined together by glycoside bonds. Typically such polysaccharides, comprise at least 40, for example at least 100, such as at least 200, including at least 500, for example at least 1000, such as at least 5000, for example 10000, such as at least 50000, for example 100000, monosaccharide units.

The monosaccharide units in such a polysaccharide may be joined in a chain, which may be branched or unbranched: such polysaccharides are referred to in this specification as ‘chain polysaccharides’. Alternatively, the monosaccharide units may be joined in a ring (which may have for example 10 to 200, preferably 10 to 100, more preferably 10 to 50, and most preferably 10 to 20, monosaccharide units), which may have one or more (preferably 1 or 2) side chains each comprising 1 to 6 (preferably 1 to 4, more preferably 1 or 2) monosaccharide units: such polysaccharides are referred to in this specification as ‘cyclic polysaccharides’.

In some embodiments, the polysaccharide comprises from 10 to 500000 monosaccharide units. In other embodiments, the polysaccharide comprises from 100 to 1000 monosaccharide units. In other embodiments, the polysaccharide comprises from 1000 to 10000 monosaccharide units. In other embodiments, the polysaccharide comprises from 10000 to 100000 monosaccharide units. In some embodiments, the polysaccharide comprises from 40 to 3000, preferably 200 to 2500, monosaccharide units.

Examples of such polysaccharides include starch and derivatives thereof (such as cationic or anionic, oxidised or phosphated starch), amylose, amylopectin, glycogen, cellulose or a derivative thereof (such as carboxymethyl cellulose), alginic acid or a salt or derivative thereof, polydextrose, pectin, pullulan, carrageenan, locust bean gum and guar and derivatives thereof (such as cationic or anionic guar).

In one embodiment, the polysaccharide comprises starch. Starches are glucose polymers in which glucopyranose units are bonded by a-linkages. It is made up of a mixture of amylose and amylopectin. Amylose consists of a linear chain of several hundred glucose molecules linked together by 1,4′-α-glycoside linkages. In contrast amylopectin is a branched molecule made of several thousand glucose units, the main chain comprising 1,4′-α-glycoside linkages but having 1,6′-α-glycoside branches approximately every 25 glucose units.

In one embodiment, the polysaccharide comprises glycogen. Glycogen is a polysaccharide that is found in animals and is composed of a branched chain of glucose residues.

In one embodiment, the polysaccharide comprises cellulose. Cellulose is a polymer formed from several thousand glucose units bonded together by 1,4′-β-glycoside linkages.

Preferred sources of the monosaccharide moiety include sucrose, maltose and maltodextrin. A particularly preferred source of the monosaccharide moiety is sucrose.

Examples of transglycosidases known in the art, together with the higher saccharides capable of being hydrolyzed by such transglycosidases, (and therefore potential monosaccharide sources for transfer of a monosaccharide moiety), are listed in Table 2 below.

TABLE 2

| | Enzyme | |
|---|---|---|
| Substrate | source | Reference |
| Maltose, nigerose, kojibiose | Aspergillus | McCleary, B. V., and T. S. |
| | niger | Gibson. 1989. Carbohydr. |
| | | Res.1185: 147-162 |
| Maltose | Aspergillus | Pazur, J. H., and D. French. |
| | oryzae | 1952. J. Bio. Chem. |
| | | 251: 265-271 |
| Maltose, maltopentaose, | Talaromyces | US 5773256 |
| maltodextrin, soluble starch, | duponti | |
| Maltodextrin (chain length 2-5 | Aspergillus | Kato, N., et al 2002. Appl |
| glucose units), isomaltose, | nidulans | Environ Microbiol. 68. |
| nigerose, kojibiose, trehalose | | 1250-1256 |
| Sucrose, trehalulose, | Aspergillus | EP 622487B1 |
| melezitose | niger | |

In a further embodiment, the monosaccharide source may be a monosaccharide phosphate. Such compounds may be formed, for example, by phosphotransferase-catalysed reaction of adenosine triphosphate (ATP) with a monosaccharide, as described below.

In one embodiment the source of monosaccharide moiety and/or the lipid is preferably not wheat flour.

In one embodiment the ratio of monosaccharide source to lipid is sufficient to provide a ratio of 1:1 monosaccharide:lipid in the reaction mixture. Each monosaccharide may be transfered to one lipid to form one glycolipid. Therefore for each hydrolysis reaction on the monosaccharide source one glycolipid molecule can be formed from one lipid molecule.

In another embodiment excess monosaccharide is provided compared to the amount of lipid so that the ratio of monosacaccharide:lipid is greater than 1:1. Excess monosacchride source may be used to drive the reaction equilibrium towards production of glycolypid.

Preferably the ratio of monosaccharide:lipid is at least 1:1, preferably at least 1.5:1, at least 2:1 at least 2.5:1, or at least 3:1.

Additional monosaccharide source may be added at different time points during the reaction to ensure that monosaccharide is in excess and the reaction equilibrium favours production of glycolipid throughout the reaction.

In another embodiment excess lipid is provided compared to the amount of monosaccharide so that the ratio of monosacaccharide:lipid is less than 1:1.

Preferably the ratio of monosaccharide:lipid is less than 1:1, preferably less than 0.6:1, less than 0.5:1 less than 0, 4:1, or less than 0.3:1.

In one embodiment the source of monosaccharide moiety may be added in an amount of 10% to 20% by weight of flour, such as, e.g. 8% to 12% by weight of flour (baker's percentages).

### Lipid Acceptor

In the present invention the lipid may be a fat-soluble (lipophilic), naturally-occurring or synthetic molecule, having a free hydroxyl (—OH) group capable of forming a glycoside bond to the transferred monosaccharide moiety. Examples of suitable lipids include fatty acids, fatty alcohols, mono- and polyglycerolipids (especially monoglycerides and diglycerides), glycolipids, phospholipids, and the like. It is intended within the scope of the present invention that the lipid acceptor may comprise a mixture of lipids.

The lipid in the present invention may not be a triglyceride, such as rape seed oil or butter. Triglycerides are not acceptor molecules for the transfer reaction of the present invention. Triglycerides can be partially hydrolysed, for example by a lipase, to form an acceptor molecule (lipid) that can be used in the transfer reaction of the present invention.

By ‘lipophilic’ means soluble in non-polar organic solvents. Preferably, such non-polar organic solvents have one or more of the following properties:
(a) a low dielectric constant (for example, a dielectric constant less than 20, preferably less than 10) and/or
(b) a weak or zero dipole moment (for example, a dipole moment of less than 1 D, preferably less than 0.5 D); and/or
(c) an absence of or substantially no hydrogen-bonding groups (O—H and/or N—H).

Examples of such non-polar organic solvents include aliphatic hydrocarbons such as pentane, hexane, heptane, alicylic hydrocarbons such as cyclohexane, aromatic hydrocarbons such as benzene, toluene or xylene, ethers such as diethyl ether, and halogenated hydrocarbons such as dichloromethane, trichloromethane (chloroform) and 1,2-dichloroethane.

Typically, the lipid includes one or more straight- or branched chain, saturated or unsaturated, hydrocarbyl (for example, alkyl, alkenyl or alkynyl) groups having at least 4 carbon atoms, preferably 6 carbon atoms, such as at least 10 carbon atoms, for example at least 12, at least 14, at least 16, at least 18, at least 20 or at least 22 carbon atoms. Preferably, such a hydrocarbyl group is an alkyl group. Alternatively, such a hydrocarbyl group comprises an alkenyl group, which may have, for example, 1 to 5 double bonds, preferably 1, 2 or 3 double bonds.

In one embodiment, the lipid includes one or more straight- or branched chain, saturated or unsaturated, hydrocarbyl (for example, alkyl, alkenyl or alkynyl) groups having 4 to 40 carbon atoms, preferably 6 to 40 carbon atoms, such as at least 10 to 40 carbon atoms, for example 12 to 40, such as 14 to 40, 16 to 40, 18 to 40, 20 to 40 or 22 to 40 carbon atoms, more preferably 10 to 24, especially 12 to 22, particularly 14 to 18, for example 16 or 18 carbon atoms. Preferably, such a hydrocarbyl group is an alkyl group. Alternatively, such a hydrocarbyl group comprises an alkenyl group, which may have, for example, 1 to 5 double bonds, preferably 1, 2 or 3 double bonds.

In one embodiment, the lipid includes one or more straight- or branched chain, saturated or unsaturated, acyl groups, ie groups of the formula R—C(═O)— wherein R is a hydrocarbyl group. Typically, such acyl groups have a total of 4 to 40 carbon atoms, preferably 6 to 40 carbon atoms, such as at least 10 to 40 carbon atoms, for example 12 to 40, such as 14 to 40, 16 to 40, 18 to 40, 20 to 40 or 22 to 40 carbon atoms, more preferably 10 to 24, especially 12 to 22, particularly 14 to 18, for example 16 or 18 carbon atoms. In one particular embodiment, such an acyl group is an alkanoyl group. Alternatively, such an acyl group comprises an alkenoyl group, which may have, for example, 1 to 5 double bonds, preferably 1, 2 or 3 double bonds.

Examples of acyl groups include saturated acyl groups such as butanoyl (butyryl), hexanoyl (caproyl), octanoyl (caprylyl), decanoyl (capryl), dodecanoyl (lauroyl), tetradecanoyl, (myristoyl), hexadecanoyl (palmitoyl), octadecanoyl (stearoyl), eicosanoyl (arachidonyl), docosanoyl (behenoyl) and tetracosanoyl (lignoceroyl) groups, and unsaturated acyl groups such as cis-tetradec-9-enoyl (myristoleyl), cis-hexadec-9-enoyl (palmitoleyl), cis-octadec-9-enoyl (oleyl), cis cis-9,12-octadecadienoyl (linoleyl), cis,cis,cis-9,12,15-octadecatrienoyl (linolenyl), and cis,cis,cis,cis-5,8,11,14-eicosa-tetraenoyl (arachidonyl) groups.

Some typical classes of lipids suitable as acceptors in the present invention are described below.

In a preferred embodiment, the lipid is a mono- or polyglycerolipid. In this specification the term ‘mono- or polyglycerolipid’ is defined as a compound comprising one or more glycerol moieties covalently bound via ester linkages to one or more acyl groups (and optionally to further additional groups such as sugar moieties, as defined and exemplified herein). The typical and preferred lengths of the acyl chains are defined and exemplified above.

The mono- or polyglycerolipid must possess at least one free hydroxyl (—OH) group to enable monosaccharide transfer to take place. It is preferred in the present invention that the free hydroxyl group is present on the glycerol portion of the molecule. However, mono- or polyglycerolipids having one or more free hydroxyl groups on a side chain (such as an attached sugar moiety) are also envisaged to be within the scope of the present invention.

In a preferred embodiment, the lipid comprises one glycerol moiety covalently bound via ester linkages to one or more acyl groups, the typical and preferred lengths of which are defined and exemplified above. The glycerol moiety may optionally also be bonded to additional groups such as sugar moieties, as defined and exemplified herein. Such compounds are referred to in this specification as ‘monoglycerolipids’ or simply ‘glycerolipids’. Glycerolipids are composed mainly of mono-, di- and tri-substituted glycerols, the most well-known being the fatty acid esters of glycerol (triacylglycerols), also known as triglycerides.

Additional subclasses are represented by glycosylglycerols, which are characterized by the presence of one or more sugar residues attached to glycerol via a glycosidic linkage. In this regard, the sugar residue may be a monosaccharide, a disaccharide, an oligosaccharide or a polysaccharide, as defined and exemplified herein. It is therefore envisaged within the scope of the present invention that the transglycosidase is capable of transferring a further monosaccharide moiety to a glycerolipid already bonded to a monosaccharide (ie a monoglycosylglyceride) to form a glycerolipid bonded to two monosaccharides (ie a diglycosylglyceride). In this regard, the two monosaccharides may be bonded to two separate OH groups on the glycerol backbone, or may be bonded to each other to comprise a disaccharide moiety attached to one OH group on the glycerol moiety.

In an alternative embodiment, the lipid is a diglycerolipid. In this specification, the term ‘diglycerolipid’ means a lipid having two glycerol moieties covalently bound to one another via ether linkages (ie a diglycerol), at least one of the glycerol moieties being covalently bound via ester linkages to one or more acyl groups, the typical and preferred lengths of which are defined and exemplified above. In this regard, the two glycerol moieties may be bonded by the ether linkage of any of the possible groups: examples of diglycerol units include α,α′-diglycerol, α,β-diglycerol, β,β-diglycerol and cyclic diglycerols, illustrated below, of which α,α′-diglycerol is preferred. The lipid may optionally comprise further additional groups such as sugar moieties, as defined and exemplified herein.

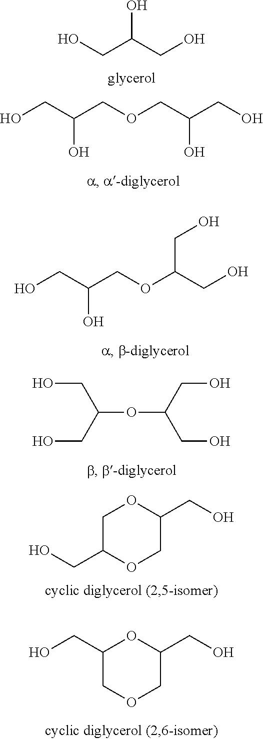

In an alternative embodiment, the lipid is a polyglycerolipid. In this specification, the term ‘polyglycerolipid’ means a lipid having more than two (preferably, 3 to 10, more preferably 3 or 4) glycerol moieties covalently bound to one another via ether linkages, at least one of the glycerol moieties being covalently bound to one or more acyl groups, the typical and preferred lengths of which are defined and exemplified above. In this regard, the glycerol moieties may be bonded by the ether linkage of any of the possible groups. The lipid may optionally comprise further additional groups such as sugar moieties, as defined and exemplified herein.

In a preferred embodiment, the lipid acceptor is a monoglyceride or a diglyceride. In this specification the term ‘monoglyceride’ (also known as monoacylglycerol) means a compound comprising one acyl group covalently bonded to a single glycerol moiety via an ester linkage (the other two OH groups of the glycerol part being free to form a glycosidic bond with the monosaccharide). Similarly, in this specification the term ‘diglyceride’ (also known as diacylglycerol) means a compound comprising two acyl groups covalently bonded to a glycerol molecule via ester linkages (the other OH group of the glycerol part being free to form a glycosidic bond with the monosaccharide).

The acyl groups of such mono- and di-glycerides may be the same or different. In addition, it is envisaged within the scope of the present invention that the lipid acceptor may comprise a mixture of mono- and/or di-glycerides.

The acyl group(s) may be present on any of the three carbons of the glycerol molecule: it is therefore envisaged within the scope of the present invention that the lipid acceptor may comprise a 1-monoacylglycerol, a 2-monoacylglycerol, a 1,2-diacylglycerol or a 1,3-diacylglycerol.

The acyl groups of suitable mono- and di-glycerides may have from 4 to 40 carbon atoms, preferably 6 to 40 carbon atoms, such as 8 to 30 carbon atoms, in particular 10 to 24 carbon atoms, for example 10, 12, 14, 16, 18, 20, 22 or 24 carbon atoms. Preferred acyl groups have 10 to 22, preferably 14 to 18, carbon atoms. The acyl groups may each be independently straight- or branched chain.

The acyl groups of suitable mono- and di-glycerides may be saturated or unsaturated. Unsaturated acyl groups may have, for example, 1 to 5 double bonds, preferably 1, 2 or 3 double bonds.

In another embodiment, the lipid acceptor is a glycoglycerolipid (also known as a glycosylglyceride). In this specification the term ‘glycoglycerolipid’, when used to define the lipid acceptor molecule, means a lipid comprising a single glycerol moiety covalently bound via ester linkages to one or more acyl groups (the typical and preferred lengths of which are defined and exemplified above) and having one or more monosaccharide moieties attached to the glycerol moiety via a glycosidic linkage, provided it contains at least one free hydroxyl group to enable transfer to take place. When more than one monosaccharide moiety is present on the glycoglycerolipid product, the monosaccharides may be bonded to different oxygen atoms on the glycerol backbone, may be bonded to each other to comprise a di-, oligo- or polysaccharide moiety attached to one oxygen atom on the glycerol moiety, or any combination thereof.

Fatty acyls (including fatty acids) are a diverse group of molecules synthesized by chain-elongation of an acetyl-CoA primer with malonyl-CoA or methylmalonyl-CoA groups. The fatty acyl structure represents the major lipid building block of complex lipids and therefore is one of the most fundamental categories of biological lipids. The carbon chain may be saturated or unsaturated, and may be attached to functional groups containing oxygen, halogens, nitrogen and sulfur. Examples of fatty acyls include the eicosanoids which are in turn derived from arachidonic acid which include prostaglandins, leukotrienes, and thromboxanes. Other major lipid classes in the fatty acyl category are the fatty esters and fatty amides. Fatty esters include important biochemical intermediates such as wax esters, fatty acyl thioester coenzyme A derivatives, fatty acyl thioester ACP derivatives and fatty acyl carnitines. The fatty amides include N-acyl ethanolamines such as anandamide.

Typically, the fatty acyl comprises a straight- or branched chain, saturated or unsaturated, acyl group, ie a group of the formula R—C(═O)— wherein R is a hydrocarbyl group. Typically, such acyl groups have a total of 4 to 40 carbon atoms, preferably 6 to 40 carbon atoms, such as at least 10 to 40 carbon atoms, for example 12 to 40, such as 14 to 40, 16 to 40, 18 to 40, 20 to 40 or 22 to 40 carbon atoms, more preferably 10 to 24, especially 12 to 22, particularly 14 to 18, for example 16 or 18 carbon atoms. In one particular embodiment, such an acyl group is an alkanoyl group. Alternatively, such an acyl group comprises an alkenoyl group, which may have, for example, 1 to 5 double bonds, preferably 1, 2 or 3 double bonds.

Glycerophospholipids, also referred to as phospholipids, are ubiquitous in nature and are key components of the lipid bilayer of cells, as well as being involved in metabolism and signaling. Glycerophospholipids may be subdivided into distinct classes, based on the nature of the polar headgroup at the sn-3 position of the glycerol backbone in eukaryotes and eubacteria or the sn-1 position in the case of archaebacteria. Examples of glycerophospholipids found in biological membranes are phosphatidylcholines (also known as PC or GPCho, and lecithin), phosphatidylethanolamines (PE or GPEtn), phosphatidylserine (PS or GPSer), phosphadityl inositol, lysophosphatidylcholines, lysophosphatidylethanolamines, N-acyl phosphatidylethanolamines and N-acyl lysophosphatidylethanolamines. In addition to serving as a primary component of cellular membranes and binding sites for intra- and intercellular proteins, some glycerophospholipids in eukaryotic cells, such as phosphatidylinositols and phosphatidic acids are either precursors of, or are themselves, membrane-derived second messengers. Typically one or both of these hydroxyl groups are acylated with long-chain fatty acids (the number of carbon atoms in the chains typically as set out above), but there are also alkyl-linked and 1Z-alkenyl-linked (plasmalogen) glycerophospholipids, as well as dialkylether variants in prokaryotes.

Sterol lipids, such as cholesterol and its derivatives are an important component of membrane lipids, along with the glycerophospholipids and sphingomyelins. The steroids, which also contain the same fused four-ring core structure, have different biological roles as hormones and signaling molecules. The C18 steroids include the estrogen family whereas the C19 steroids comprise the androgens such as testosterone and androsterone. The C21 subclass includes the progestogens as well as the glucocorticoids and mineralocorticoids. The secosteroids, comprising various forms of vitamin D, are characterized by cleavage of the B ring of the core structure. Other examples of sterols are the bile acids and their conjugates, which in mammals are oxidized derivatives of cholesterol and are synthesized in the liver.

Saccharolipids describe compounds in which fatty acids are linked directly to a sugar backbone, forming structures that are compatible with membrane bilayers. In the saccharolipids, a sugar substitutes for the glycerol backbone that is present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo₂-Lipid A, a hexa-acylated disaccharide of glucosamine that is glycosylated with two 3-deoxy-D-manno-octulosonic acid (Kdo) residues.

In an alternative embodiment, the lipid acceptor is a lysophospholipid. A lysophospholipid comprises a glycerol moiety having only one acyl group (as defined and exemplified above) covalently bonded to a glycerol oxygen atom via an ester linkage and a phosphate group covalently bonded to another glycerol oxygen atom to form a phosphate ester: the said lysophospholipids therefore possess a free OH group on the remaining glycerol carbon atom. Suitable lysophospholipids include lysophosphatidylcholines (also known as lyso-PC), lysophosphatidylethanolamines (PE or GPEtn), phosphatidylserine (PS or GPSer), phosphadityl inositol, lysophosphatidylcholines, lysophosphatidylethanolamines, N-acyl phosphatidyl-ethanolamines and N-acyl lysophosphatidyl-ethanolamines. The lysophospholipid may be formed in situ by hydrolysis of one of the ester linkages on the corresponding phospholipid.

In one embodiment the source of monosaccharide moiety and/or the lipid is preferably not wheat flour.

In one embodiment the ratio of monosaccharide source to lipid is sufficient to provide a ratio of 1:1 monosaccharide:lipid in the reaction mixture. Each monosaccharide may be transfered to one lipid to form one glycolipid. Therefore for each hydrolysis reaction on the monosaccharide source one glycolipid molecule can be formed from one lipid molecule.

In another embodiment excess monosaccharide is provided compared to the amount of lipid so that the ratio of monosacaccharide:lipid is greater than 1:1. Excess monosacchride source may be used to drive the reaction equilibrium towards production of glycolypid.

Preferably the ratio of mono-saccharide:lipid is at least 1:1, preferably at least 1.5:1, at least 2:1 at least 2.5:1, or at least 3:1.

Additional monosaccharide source may be added at different time points during the reaction to ensure that mono-saccharide is in excess and the reaction equilibrium favours production of glycolipid throughout the reaction.

In another embodiment excess lipid is provided compared to the amount of monosaccharide so that the ratio of monosacaccharide:lipid is less than 1:1.

Preferably the ratio of monosaccharide:lipid is less than 1:1, preferably less than 0.6:1, less than 0.5:1 less than 0.4:1, or less than 0.3:1.

In one embodiment the lipid is added in an amount of up to 10% by weight of flour, such as, e.g. up to 3, up to 1.5, or up to 1% by weight of flour (baker's percentages).

### Glycolipid Product

The transglycosidase-mediated transfer of one or more monosaccharide moieties to a lipid generates a glycolipid product. In this specification the term ‘glycolipid’ includes any lipid (as defined and exemplified above) having one or more monosaccharide moieties covalently bonded to an oxygen atom on the lipid (preferably, although not exclusively, via a glycoside bond). When more than one monosaccharide moiety is present on the glycolipid product, the monosaccharide moieties may be bonded directly to separate oxygen atoms on the lipid. Alternatively and/or additionally, the monosaccharide moieties may be bonded to each other to comprise a di-, oligo- or polysaccharide moiety attached to one oxygen atom on the lipid.

In a preferred embodiment, the glycolipid product is a glycoglycerolipid (also known as a glycosylglyceride). In this specification the term ‘glycoglycerolipid’, when used to define the product, means a lipid comprising a single glycerol moiety covalently bound via ester linkages to one or more acyl groups (the typical and preferred lengths of which are defined and exemplified above) and having one or more monosaccharide moieties attached to the glycerol moiety via a glycosidic linkage. The product may (and typically does) have a free hydroxyl group, preferably on one or more of the monosaccharide moieties. When more than one monosaccharide moiety is present on the glycoglycerolipid product, the monosaccharides may be bonded to different oxygen atoms on the glycerol backbone, may be bonded to each other to comprise a di-, oligo- or polysaccharide moiety attached to one oxygen atom on the glycerol moiety, or any combination thereof. It is therefore envisaged within the scope of the present invention that the transglycosidase enzyme may transfer a further monosaccharide moiety to a glycoglycerolipid substrate to produce another glycoglycerolipid having an additional monosaccharide moiety.

In an alternative embodiment, the lipid is a glycodiglycerolipid. In this specification, the term ‘glycodiglycerolipid’ means a lipid having two glycerol moieties covalently bound to one another via ether linkages (ie a diglycerol), wherein at least one of the glycerol moieties are covalently bound via ester linkages to one or more acyl groups, (the typical and preferred lengths of which are defined and exemplified above) and having one or more monosaccharide moieties covalently bonded to an oxygen atom on the diglycerol backbone via a glycosidic linkage. The two glycerol moieties which form the diglycerol backbone may be bonded by the ether linkage of any of the possible groups: examples of diglycerol units include α,α′-diglycerol, α,β-diglycerol, β,β-diglycerol and cyclic diglycerols, illustrated above, of which α,α′-diglycerol is preferred.

In another embodiment, the glycolipid is a glycopolyglycerolipid. In this specification the term ‘glycopolyglycerolipid’ is defined as a compound comprising more than two (preferably, 3 to 10, more preferably 3 or 4) glycerol moieties covalently bound to one another via ether linkages, wherein at least one of the glycerol moieties are covalently bound to one or more acyl groups (the typical and preferred lengths of which are defined and exemplified above) and having one or more monosaccharide moieties covalently bonded to an oxygen atom on the polyglycerol backbone via a glycosidic linkage. In this regard, the glycerol moieties may be bonded by the ether linkage of any of the possible groups.

In preferred embodiments, the transfer of the monosaccharide moiety to the mono- or diglyceride generates a product selected from a monoglucosyl monoglyceride, a polyglucosyl monoglyceride (for example, a diglucosyl monoglyceride, a triglucosyl monoglyceride), a monoglucosyl diglyceride, a polyglucosyl diglyceride (eg a diglucosyl diglyceride, a triglucosyl diglyceride) or a mixture thereof. Where more than one monosaccharide moiety is transferred to the lipid acceptor, the monosaccharide moieties may be bonded to the glycerol oxygen atoms, to another monosaccharide, or any combination thereof, as described above.

In one embodiment, the glycolipid product (preferably a glycoglycerolipid) is isolated from the reaction mixture. Isolation of the glycolipid product from the reaction mixture permits the product to be used in a variety of applications, in particular the purified or isolated glycolipid may be added in some foodstuffs and some laundry and cleaning applications, avoiding contamination and possible side-reactions by the other reactants and products of the glycosyl transfer reaction. The product may be isolated from the reaction mixture by one or more of a number of conventional techniques, including solvent extraction, distillation, crystallisation, washing and precipitation.

In one embodiment, the glycolipid product (preferably a glycoglycerolipid) is purified following (or as an alternative to) isolation from the reaction mixture. Purification of the glycolipid product from the reaction mixture improves the quality of the product and makes it more suitable for use in a variety of applications, in particular the purified or isolated glycolipid may be added in some foodstuffs and some laundry and cleaning applications. The product may be purified by one or more of a number of conventional techniques, including chromatography, solvent extraction, precipitation, distillation and crystallisation.

Isolated and/or Purified

In one aspect, preferably the glycolipid product according to the present invention is in an isolated form. The term “isolated” means that the glycolipid product is at least substantially free from at least one other component with which the glycolipid product is associated in the reaction mixture.

In one aspect, preferably the glycolipid product according to the present invention is in a purified form. The term “purified” means that a given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### In Situ Production of Glycolipids

In a preferred aspect of the invention, the glycosylation takes place in situ in a composition, in particular a food product or food product intermediate composition (as defined below) or a laundry composition. Preferably, at least one of the transglycosidase enzyme, monosaccharide source and lipid are added to the remaining components of the composition. More preferably two of these ingredients are added, and most preferably, all three are added to the composition. However, it is also envisaged within the scope of the present invention that one or more of the remaining components of the composition may partially or wholly act as the monosaccharide source, the lipid, or both.

### In Situ Production in Food

Therefore, the invention comprises in a further aspect a method for in situ production of a glycosylated lipid in a food product or food product intermediate composition, the method comprising contacting the food product or food product intermediate with a source of monosaccharide moiety, as defined herein, a lipid, as defined herein, and a transglycosidase enzyme, as defined herein.

In this aspect of the invention, the food product is preferably a baked product (as defined below). Furthermore, the food product intermediate is preferably a dough.

In this aspect of the invention, the source of monosaccharide moiety and/or the lipid is preferably not wheat flour or malt extract.

In this aspect of the invention (as well as other baking applications described below), the source of monosaccharide moiety is preferably sucrose. Sucrose is a more applicable donor substrate in baking applications than maltose, as sucrose is much cheaper than maltose. Furthermore, without wishing to be bound by theory, it is believed that the fermentation of sucrose by yeast is more stable than that of maltose.

In some aspects of the present invention excess source of monosaccharide, for example sucrose, may be added to the foodstuff to act as a sweetener in the foodstuff as well as to provide a sufficient monosaccharide source for the glycosylation reaction. In these aspects the amount of monosaccharide source added to the foodstuff or foodstuff intermediate should be enough to provide sufficient monosaccharide source for the glycosylation reaction and to make the foodstuff sufficiently sweet. The amount of monosaccharide source added may therefore depend on how sweet the foodstuff is intended to be.

Accordingly, In this aspect of the invention, the source of monosaccharide moiety may be added in an amount of up to 30%, such as, e.g. up to 25%, 20%, 15%, 12%, 8% or 5% by weight (baker's percentage). In some aspects of the invention, the monosaccharide source is added in an amount of from about 10% by weight to about 20% by weight, such as, e.g., from about 8% by weight to about 12% by weight. In some aspects the source of monosaccharide moiety may be less than 5% by weight.

In this aspect of the invention the lipid is preferably selected from a monoglyceride or a diglyceride. The lipid may be added in an amount of up to 10%, such as, e.g. 3%, 1.5% or 1% by weight (baker's percentage). In some aspects the added lipid may be less than 1% by weight.

Percentages of ingredients in bakery products can be expressed in Baker's percentage. Baker's percentage means that the weight of the flour equals 100%. All the other ingredients are calculated in proportion to the weight of flour. Weight of ingredient divided by weight of total flour, times 100=ingredient %. If there is more than one type of flour the total weight of all types of flour added together adds up to 100%.

### In Situ Use in Laundry Compositions

The invention comprises in a further aspect a method for in situ production of a glycosylated lipid in a laundry composition, the method comprising contacting the transglycosidase enzyme, as defined herein with a source of monosaccharide moiety as defined herein and a lipid as defined herein.

In this aspect of the invention the laundry composition comprises one or more of a transglycosidase enzyme, a monosaccharide source and/or a lipid.

The laundry composition may further comprise a lipase (E.C. 3.1.1). The laundry composition may further comprise a stain comprising one or more of a transglycosidase enzyme, a monosaccharide source and/or a lipid. The stain may comprise a triglyceride and/or a diglyceride and/or a monoglyceride. The stain may be on a surface, for example a fabric, the laundry composition may therefore comprise a surface for example a fabric.

In one embodiment a laundry composition comprises a transglycosidase enzyme as defined herein, a monosaccharide source and a stain comprising diglycerides and/or monoglycerides. The monosaccharide moiety is transferred by the transglycosidase enzyme to the lipid and a glycolipid is produced.

In one embodiment the stain comprises a triglyceride and the laundry composition further comprises a lipase. Hydrolysis of the triglyceride by the lipase provides a source of diglycerides and/or monoglycerides. A monosaccharide moiety is transferred by the transglycosidase enzyme to the diglyceride and/or monoglyceride to form a glycolipid.

In one embodiment converting a triglyceride, diglyceride or a monoglyceride into a glycolipid helps remove a stain comprising a lipid from a fabric.

In one embodiment a glycolypid, either added or produced in situ in the laundry composition helps remove a stain comprising a lipid from a fabric.

### Combinations

The transglycosidase enzyme (in particular, the transglucosidase enzyme) may be used according to the present invention in combination with one or more further active agents. Such combinations may offer advantages, including synergy, when used together in a composition, in particular a foodstuff.

In particular, the transglycosidase enzyme (in particular, the transglucosidase enzyme) may be used according to the present invention in combination with one or more further enzymes as active agents. Such combinations may offer advantages, including synergy, when used together in a composition, in particular a foodstuff.

In one embodiment, the further enzyme is another transglycosidase enzyme (in particular, a transglucosidase enzyme), so that two (or more) different transglycosidase (particularly transglucosidase) enzymes are used in combination. Without wishing to be bound by theory, it is envisaged that one transglucosidase may catalyse the transfer of one glucose moiety to a monoglyceride acceptor and another may catalyse the transfer to the glucosyl moiety on the resultant glycosylmonoglyceride thereby elongating the glucan chain on the monoglyceride.

In one embodiment, the further enzyme is a glycosidase (E.C. 3.2.1). Without wishing to be bound by theory, it is envisaged that combining a glycosidase with the transglycosidase enzyme of the present invention may be particularly advantageous in that the glycosidase is capable of hydrolysing glycoside bonds of longer-chain higher saccharides to shorter-chain higher saccharides (especially di- and oligosaccharides), the monosaccharide moieties of which can then be more easily transferred to a lipid than from such longer-chain higher saccharides. In particular, the glycosidase may comprise an amylase, such as α-amylase (E.C. 3.2.1.1) or β-amylase (E.C. 3.2.1.2). Such amylase enzymes are capable of hydrolysing starch to shorter-chain oligosaccharides such as maltose: the glucose moiety can then be more easily transferred from maltose to a lipid than from the original starch molecule.

In one embodiment, the further enzyme is a hexosyltransferase (E.C. 2.4.1). Without wishing to be bound by theory, it is envisaged that combining a hexosyltransferase with the transglycosidase enzyme of the present invention may be particularly advantageous in that the hexosyltransferase is capable of transferring monosaccharide moieties from compounds on which the transglycosidase enzyme of the present invention is generally inactive to form other compounds, such as mono- or higher saccharides (especially di- and oligosaccharides) on which the transglycosidase enzyme of the present invention can act to transfer the monosaccharide moieties to the lipid. In addition, without wishing to be bound by theory, it is envisaged that glucosyltransferases and other hexosyltransferases could transfer one or more glucosyl moieties to the glucosyl moiety or moieties already present on or previously transferred to the lipid by the transglycosidase of the present invention, thereby elongating the glucan chain on the lipid.

In another embodiment, the further enzyme is a carboxylic ester hydrolase (E.C. 3.1.1). Without wishing to be bound by theory, it is envisaged that combining a carboxylic ester hydrolase with the transglycosidase enzyme of the present invention combination may be particularly advantageous in that the carboxylic ester hydrolase is capable of partially hydrolysing a triglyceride (which lacks the necessary free OH group to accept a monosaccharide moiety) into a mono- or diglyceride which are preferred as lipid acceptor molecules in the present invention. In particular, the carboxylic ester hydrolase may comprise a carboxylesterase (E.C. 3.1.1.1).

In a yet further embodiment, the further enzyme is a phosphotransferase (E.C. 2.7.1). Without wishing to be bound by theory, it is envisaged that combining a phosphotransferase with the transglycosidase enzyme of the present invention may be particularly advantageous in that a negatively charged glucoglycerolipid could be generated as an end product with modified properties. The phosphotransferase could transfer to a glucose moiety on e.g maltose and then subsequently be transferred to the lipid by the transglycosidase generating a charged emulsifier. Alternatively a phosphotransferase could transfer phosphate to the glucoglycerolipid itself. Alternatively the phosphate group could be transferred to another free OH on the glycerol backbone. Particularly preferred examples of phosphotransferases are those classified in E.C. 2.7.1.61, 2.7.1.63, 2.7.1.79 and 2.7.1.142.

Examples of further classes of enzymes suitable for combination with the transglycosidase in the present invention include oxidases (E.C. 1.1.3) and O-acyltransferases (particularly those classed in E.C. 2.3.1.43).

In one embodiment the laundry composition of the present invention may comprise a transglycosidase enzyme of the present invention in combination with one or more other enzymes, such as a protease, an amylase, a glucoamylase, a maltogenic amylase, a lipase, a cutinase, a carbohydrase, a cellulase, a pectinase, a mannanase, an arabinase, a galactanase, a xylanase, an oxidase, a laccase, and/or a peroxidase, and/or combinations thereof. In general the properties of the chosen enzyme(s) should be compatible with the selected detergent, (e.g., pH-optimum, compatibility with other enzymatic and non-enzymatic ingredients, etc.), and the enzyme(s) should be present in effective amounts.

Proteases: suitable proteases include those of animal, vegetable or microbial origin. Chemically modified or protein engineered mutants are also suitable. The protease may be a serine protease or a metalloprotease, e.g., an alkaline microbial protease or a trypsin-like protease. Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115. Suitable commercially available protease enzymes include Alcalase®, Savinase®, Esperase®, and Kannase™ (Novozymes, formerly Novo Nordisk A/S); Maxatase®, Maxacal™, Maxapem™, Properase™, Purafect®, Purafect OxP™, FN2™, and FN3™ (Genencor International, Inc.).

Lipases: The further enzyme may be a lipase (EC 3.1.1) capable of hydrolysing carboxylic ester bonds to release carboxylate. Examples of lipases include but are not limited to triacylglycerol lipase (EC 3.1.1.3), galactolipase (EC 3.1.1.26), phospholipase A1 (EC 3.1.1.32, phospholipase A2 (EC 3.1.1.4) and lipoprotein lipase A2 (EC 3.1.1.34). More specifically, suitable lipases include lipases from Mucor miehei, F. venenatum, H. lanuginosa, Rhizomucor miehei candida antarctica, F. oxysporum, glycolipase from Fusarium heterosporum (such as GRINDAMYL™ POWERBake 4050 (Danisco A/S)) and variants, homologues and derivatives thereof. Suitable lipases include those of bacterial or fungal origin. Chemically modified or protein engineered mutants are included. Examples of useful lipases include, but are not limited to, lipases from Humicola (synonym Thermomyces), e.g. H. lanuginosa (T. lanuginosus) (see, e.g., EP 258068 and EP 305216) and H. insolens (see, e.g., WO 96/13580); a Pseudomonas lipase (e.g., from P. alcaligenes or P. pseudoalcaligenes; see, e.g., EP 218 272), P. cepacia (see, e.g., EP 331 376), P. stutzeri (see, e.g., GB 1,372,034), P. fluorescens, Pseudomonas sp. strain SD 705 (see, e.g., WO 95/06720 and WO 96/27002), P. wisconsinensis (see, e.g., WO 96/12012); a Bacillus lipase (e.g., from B. subtilis; see, e.g., Dartois et al. (1993)), B. stearothermophilus (see, e.g., JP 64/744992), or B. pumilus (see, e.g., WO 91/16422). Additional lipase variants contemplated for use in the formulations include those described, for example, in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105. Some commercially available lipase enzymes include Lipolase® and Lipolase® Ultra (Novozymes, formerly Novo Nordisk A/S).

Polyesterases: Suitable polyesterases include, but are not limited to, those described in WO 01/34899 (Genencor International, Inc.) and WO 01/14629 (Genencor International, Inc.), and can be included in any combination with other enzymes discussed herein.

Amylases: The compositions can comprise amylases such as α-amylases (EC 3.2.1.1), β-amylases (EC 3.2.1.2) and γ-amylases (EC 3.2.1.3). These can include amylases of bacterial or fungal origin, chemically modified or protein engineered mutants are included. Commercially available amylases, such as, but not limited to, Duramyl®, Termamyl™, Fungamyl® and BAN™ (Novozymes, formerly Novo Nordisk A/S), Rapidase®, and Purastar® (Genencor International, Inc.), LIQUEZYME™, NATALASE™, SUPRAMYL™, STAINZYME™, FUNGAMYL and BAN™ (Novozymes A/S), RAPIDASE™, PURASTAR™ and PURASTAROXAM™ (from Genencor International Inc.).

Peroxidases/Oxidases: Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include GUARDZYME® (Novozymes A/S).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307; 5,648,263; 5,691,178; 5,776,757; and WO 89/09259, for example. Exemplary cellulases contemplated for use are those having color care benefit for the textile. Examples of such cellulases are cellulases described in EP 0495257; EP531372; WO 99/25846 (Genencor International, Inc.), WO 96/34108 (Genencor International, Inc.), WO 96/11262; WO 96/29397; and WO 98/08940, for example. Other examples are cellulase variants, such as those described in WO 94/07998; WO 98/12307; WO 95/24471; PCT/DK98/00299; EP 531 315; U.S. Pat. Nos. 5,457,046; 5,686,593; and 5,763,254. Commercially available cellulases include Celluzyme® and Carezyme® (Novozymes, formerly Novo Nordisk A/S); Clazinase™ and Puradax® HA (Genencor International, Inc.); and KAC-500(B)™ (Kao Corporation).

An example of a commercially available mannose is MANNAWAY™ from Novozymes, Denmark).

### Food Products

The present invention is particularly suitable for improving the emulsification properties of a food product, in particular dough and a baked product prepared from dough.

The invention therefore comprises, in an alternative aspect, a method of improving the emulsification properties of a food product, the method comprising contacting the food product or food product intermediate with a source of monosaccharide moiety, as defined herein, a lipid, as defined herein, and a transglycosidase enzyme, as defined herein (in particular a transglucosidase enzyme classified in E.C. 3.2.1.20 and/or Glycoside Hydrolase Family 31 of the Carbohydrate-Active Enzymes database).

In one aspect, the invention comprises a method of improving the emulsification properties of a food product, the method comprising contacting the food product or food product intermediate with a source of monosaccharide moiety, as defined herein, a lipid, as defined herein, and a transglucosidase enzyme, as defined herein, provided that the source of monosaccharide moiety and the lipid is not wheat flour or malt extract.

In this specification, the term “food product intermediate” is intended to mean a non-final food product which is in the process of being processed into a food product. Such a food product intermediate may comprise all or only some of the ingredients' necessary for the production of the food product, at the point where it is contacted by the source of monosaccharide moiety, lipid and transglycosidase enzyme. In one aspect the food product intermediate is a dough comprising a cereal flour, preferably a wheat flour.

The invention further comprises, in a further alternative aspect, a composition for improving the emulsification properties of a food product comprising a source of monosaccharide moiety (as defined above: in particular, a source of glucose moiety), a lipid (as defined above: in particular a mono- and/or di-glyceride), and a transglycosidase enzyme, as defined above (in particular a transglucosidase enzyme classified in E.C. 3.2.1.20 and/or Glycoside Hydrolase Family 31 of the Carbohydrate-Active Enzymes database).

The invention additionally comprises, in a yet further alternative aspect, a food product improving composition comprising a source of monosaccharide moiety (as defined above: in particular, a source of glucose moiety), a lipid (as defined above: in particular a mono- and/or di-glyceride), and a transglycosidase enzyme, as defined above (in particular a transglucosidase enzyme classified in E.C. 3.2.1.20 and/or Glycoside Hydrolase Family 31 of the Carbohydrate-Active Enzymes database).

In one aspect, the invention comprises a food product improving composition including a transglucosidase enzyme, as defined herein; a source of monosaccharide moiety, as defined herein; and a lipid, as defined herein; provided that the source of monosaccharide moiety and the lipid is not wheat flour or malt extract.

The invention also comprises, in a still further alternative aspect, a food product (in particular a dough and baked products prepared from dough) prepared from one or other of the above compositions and/or by the above method. Typically, the method further comprises, if necessary, subjecting the resulting dough to baking under suitable conditions.

For the purposes of this application, the term “bakery products” and/or “baked products” refer to products comprising cereal flour such as pasta, noodles and leavened bread products including: bread loaves, rolls or toast bread; Danish pastry; sweet dough products; laminated doughs; liquid batters; muffins, doughnuts; biscuits; cookies; crackers and cakes. For the avoidance of doubt, in one embodiment the terms “bakery products” and/or “baked products” do not include, gnocchi and couscous.

For the purposes of this application, the term “dough” refers to a dough suitable for preparing a baked product as defined above. The term dough does not include dough used for preparing products such as, gnocchi and couscous.

Dough components comprise flour, water and a leavening agent such as yeast or a conventional chemical leavening agent. It is, however, within the scope of the present invention that further optional dough components may be added to the dough mixture.

Typically, such further optional dough components include conventionally used dough components such as salt, sweetening agents such as sugars, syrups or artificial sweetening agents, lipid substances including shortening, margarine, butter or an animal or vegetable oil, glycerol and one or more dough additives such as starch, flavouring agents, lactic acid bacterial cultures, vitamins, minerals, hydrocolloids such as alginates, carrageenans, pectins, vegetable gums including e.g. guar gum and locust bean gum, and dietary fibre substances.

In bakery products and dough the proportion of different glycoglycerolipids produced in situ depends on the availability of the mono- and di-glycerides and monosaccharide source. Different glycoglycerolipids have different properties as emulsifiers. In bakery products and dough diglucosyl-monoglyceride DGMG is the strongest emulsifier followed by monoglucosyl-monoglyceride MGMG, followed by diglucosyl-diglyceride DGDG, followed by monoglucosyl-diglyceride MGDG.

The present invention will now be described by way of example only.

### EXAMPLES

### Example 1

### In Vitro Glucosylation of Monoglyceride by TGL-500

C10 based monoglycerides were tested as acceptor substrates for transglucosidase in a buffer based in vitro system.

In a total volume of 50 ml the reactions consisted of:

50 mM sodium acetate (pH 6.0)
30% (by weight) C10 monoglyceride
5% maltose (by weight)

The monoglyceride was allowed to dissolve and the solution was dispersed by Ultra Turrax treatment for 20 s. Reactions were initiated by addition of 100 U Transglucosidase L-500 (TGL-500) (Genencor, lot nr 102-04208-001) and incubated while stirring at 45° C. After 18 and 26 hours an additional 2.5 g maltose was added to the reaction to ensure that the equilibrium in the reaction mixture favoured glycolipid formation. An additional 25 U TGL-500 was added after 42 hours. Samples were taken out after 0, 18, 42 and 96 hours from the initiation of the reaction and freeze dried.

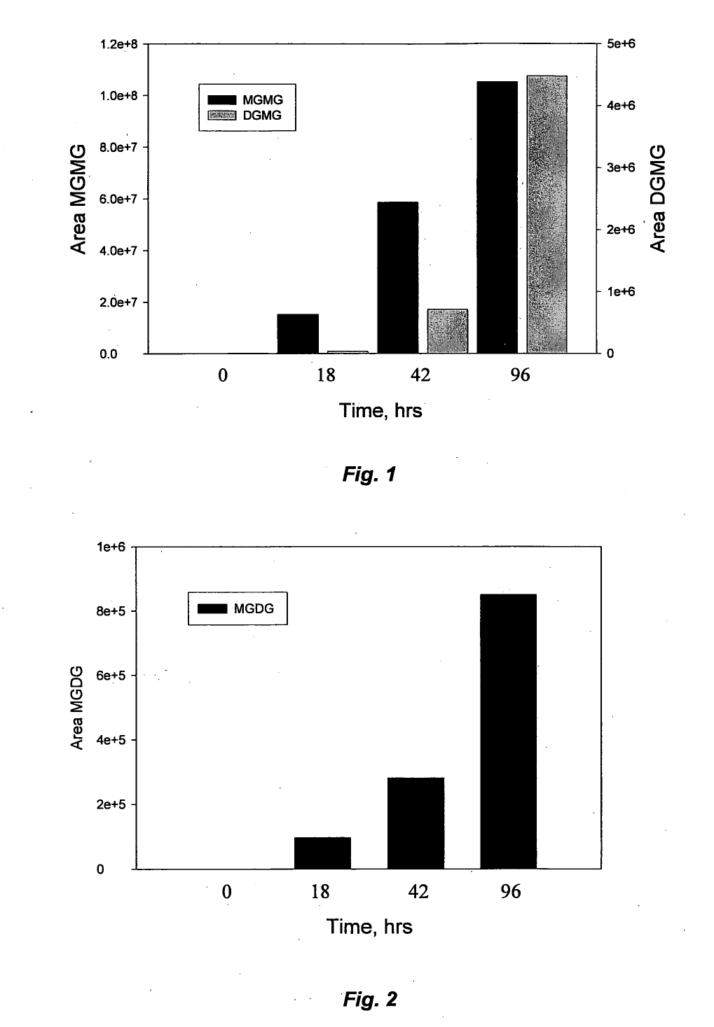

The samples were analyzed for glucoglycerolipids composition by Liquid Chromatography-mass spectrometry (LC-MS). The samples are analysed with reversed-phase high-performance liquid chromatography coupled on-line with electrospray ionisation mass spectrometry in positive mode (HPLC/ESP-MS). The column is a C18 column and the gradient is based on water/acetone. Sodium acetate was added for adduct formation in positive mode. The amounts of monoglucosyl-monoglyceride (MGMG) and diglucosyl-monoglyceride (DGMG) increased significantly over time (FIG. 1). The starting material contains 3% diglyceride and in addition to the formation of MGMG and DGMG we could identify the generation of monoglucosyl-diglyceride (MGDG) which increased over time (FIG. 2).

It can be concluded from this LC-MS analysis that the transglucosidase can transfer glucosyl units to monoglyceride generating mono- and diglucosylmonoglyceride. Furthermore, we have shown that the transglucosidase can transfer glucosyl units to a diglyceride generating glucosyldiglyceride.

### Example 2

### In Situ Generation of Glucoglycerolipids in Bakery

Glucan donor substrates for transglucosidase, TGL-500, are maltose and maltodextrin and in order to provide optimal conditions for TGL-500 maltose was tested and added to the sponge and baked according to the recipe given below. The transglucosidase, tested in this example was the Transglucosidase L-500 (TGL-500) (available from Danisco/Genencor, lot nr 102-04208-001). The bakery trial was performed according to the recipe given in Tables 3 and 4 below.

After proof the dough samples were collected together with crumb samples of the final breads and 10 g samples were freeze dried before extraction of lipids. For lipid extraction the freeze dried samples were milled in a coffee mill and passed through an 800 micron screen. 1.5 g of freeze dried sample was scaled in a 15 ml centrifuge tube with a screw lid. 7.5 ml of water saturated butanol (WSB) was added. The centrifuge tube was placed in a boiling water bath for 10 min. The tubes were placed in a Rotamix and mixed at 45 rpm for 20 min. at ambient temperature. The samples were then placed in a boiling water bath again for 10 min. then mixed on the Rotamix for a further 30 min. at ambient temperature. The tubes were centrifuged at 3500 g for 5 min. 5 ml of supernatant was transferred into a vial. WSB was evaporated to dryness under a steam of nitrogen. Lipid samples were analyzed for glucoglycerolipid composition by gas chromatography (GC). Sugar (galactosyl and glucosyl) modified monoglyceride and diglyceride were identified and quantified in the samples. Galactosyl and glucosyl modified lipids are chromatographically identical when analyzed by GC.

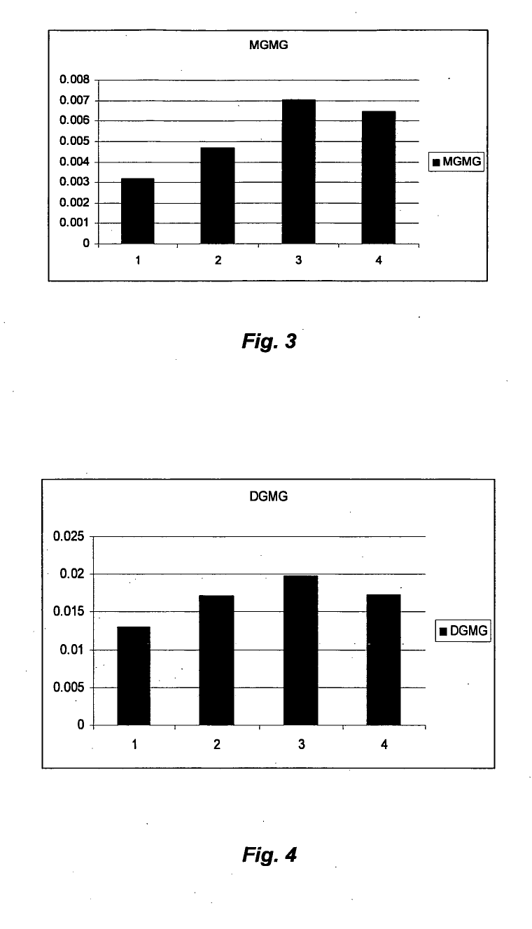

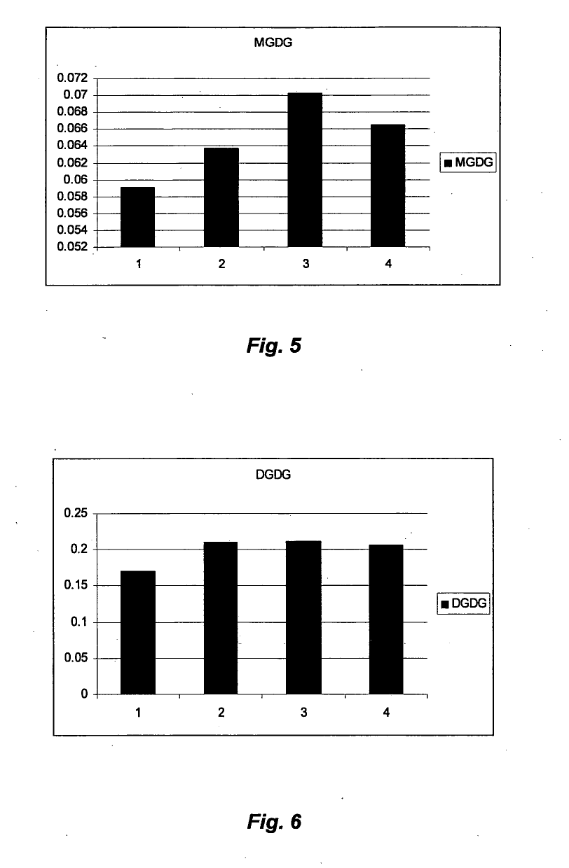

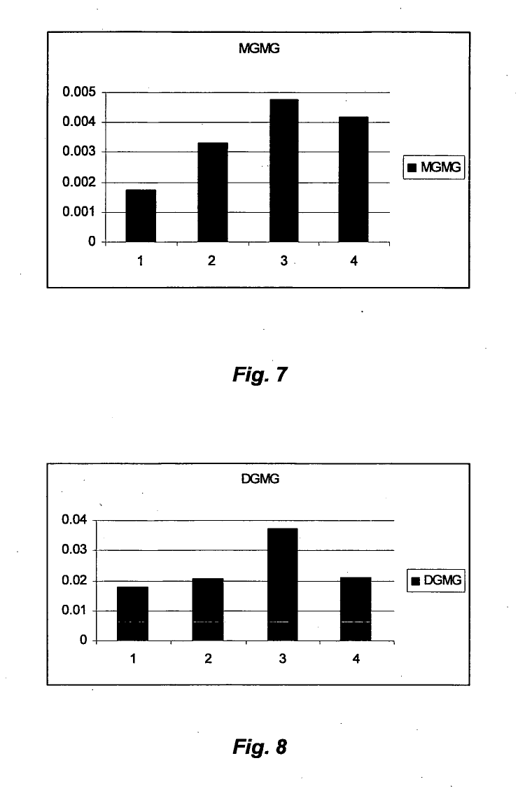

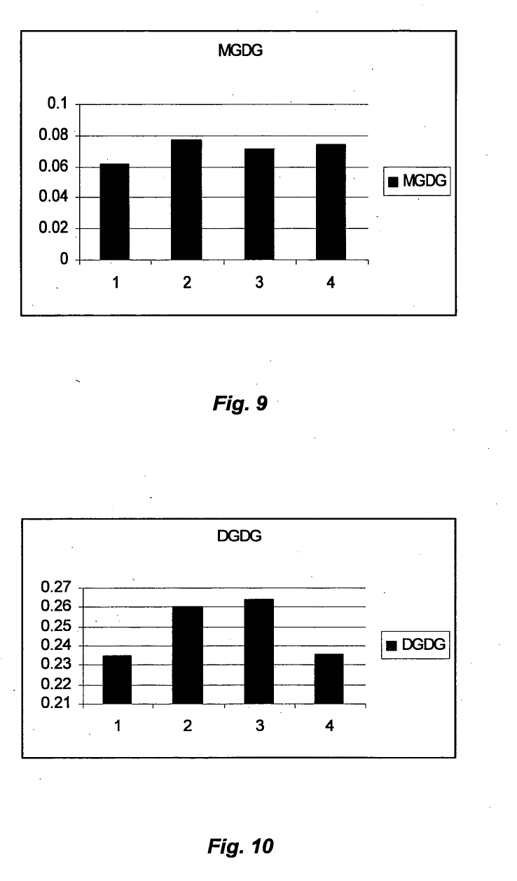

The results are shown in FIGS. 3-10 in which the following abbreviations are used:

MGMG: Mono-galactosyl/glucosyl monoglyceride
DGMG: Di-galactosyl/glucosyl monoglyceride
MGDG: Mono-galactosyl/glucosyl diglyceride
DGDG: Di-galactosyl/glucosyl diglyceride

For each the recipes numbered 1-4 in the Figures, the experimental setup was as follows:

1: No TGL-500, 8% sucrose
2: 12.5 ml/kg TGL-500, 8% sucrose
3: 12.5 ml/kg TGL-500, 8% maltose
4: 12.5 ml/kg TGL-500, 4% sucrose, 4% maltose

It can be concluded that, by applying the transglucosidase in a dough system, glucoglycerolipids (which are useful as emulsifiers) can be generated in situ. The amounts of generated glucoglycerolipids are increased when maltose is added to the dough system (setup 3 and 4). An increase, compared to setup 1, in glucoglycerolipid content is also observed in setup 2 where TGL-500 and sucrose are added.

### Recipe Used in Example 2:

TABLE 3
Sponge

| | % (baker's | |
|---|---|---|
| Ingredients | percentage) | g |
| Wheat flour (Bucchaneer: Con Agra, US) | 70 | 1400 |
| Water | 65% of total | 768 |
| | water amount | |
| Rape seed oil (Aarhus Karlshamn, | 2 | 40 |
| Denmark) | | |
| Sodium Stearoyl Lactylate (GRINDSTED | 0.375 | 7.5 |
| SSL P55 Veg) (Danisco, Denmark) | | |
| Compressed yeast (De Danske | 3 | 60 |
| Spritfabrikker, Denmark) | | |
| Distilled monoglycerides | 0.5 | 10 |
| (DIMODAN PH200: Danisco, Denmark) | | |
| Monosaccharide source | 8 | 160 |
| (Sugar: Danisco, Denmark). Maltose | | |
| (Fluka) | | |
| TGL-500 (Genencor, lot nr 102-04208- | 1.25 | 25 |
| 001) | | |

TABLE 4
Dough

| | % (baker's | |
|---|---|---|
| Ingredients | percentage) | g |
| Wheat flour (Bucchaneer: Con Agra, US) | 30 | 600 |
| Salt (NaCl) (Brøste, Denmark) | 1.5 | 30 |
| Calcium Propionate (Sigma-Aldrich) | 0.25 | 5 |
| Compressed yeast | 0.9 | 18 |
| Ascorbic Acid, (DKSK, Denmark) | 60 ppm | 0.12 |
| Water | 35 of total Water | 415 |
| | amount | |
| Azodicarbonamide | 40 ppm | 0.08 |
| (Sigma-Aldrich) | | |

### Equipment:

Mixer: Hobart (sponge); Diosna (dough)
Proofing cabinet

### Moulder: Glimek

### Oven: MIWE Roll in

### Procedure:

### Sponge:

1. Mix all ingredients 1 minute at 1^{st} speed-3 minute 2^{nd} speed on Hobart
2. Sponge temp. must be app. 24° C.
3. Ferment sponge 3 hours at 25° C., 85% relative humidity

### Dough:

1. Mix sponge and all remaining ingredients except salt for 1 min low-2 min high on Diosna
2. Add salt—mix 8 minutes high speed
3. Scale 550 g all 4 batch and mould
4. Rest dough 10 min at ambient temperature
5. Mould on Glimek: 1:4-2:3-3:15-4:12—width: 8 in both sides
6. Put dough into tins
7. Proof to height (about 65 minutes) at 43° C., 95% RH
8. Bake for 26 minutes at 200° C.
9. Take breads out of tins and cool for 70 min before weighing and measuring of volume

### Example 3

### In Vitro Glucosylation of Monoglyceride by TGL-500 Using Maltodextrin and Sucrose as Donors

C10 based monoglycerides were applied as acceptor substrates for transglucosidase in a buffer based in vitro system.

In a total volume of 50 ml the reactions consisted of:

50 mM sodium acetate (pH 6.0)
30% C10 monoglyceride (by weight)
5% maltodextrin or sucrose (by weight)

The monoglyceride was allowed to dissolve and the solution was dispersed by Ultra Turrax treatment for 20 s. Reactions were initiated by addition of 100 U TGL-500 and incubated while stirring at 45° C. After 3, 18 and 22 hours an additional 2.5 g maltodextrin or sucrose were added to the reaction to ensure that the equilibrium in the reaction mixture favoured glycolipid formation. An additional 25 U TGL-500 was added after 18 hours. Samples were taken out after 0, 20 and 120 hours from the initiation of the reaction and freeze dried.

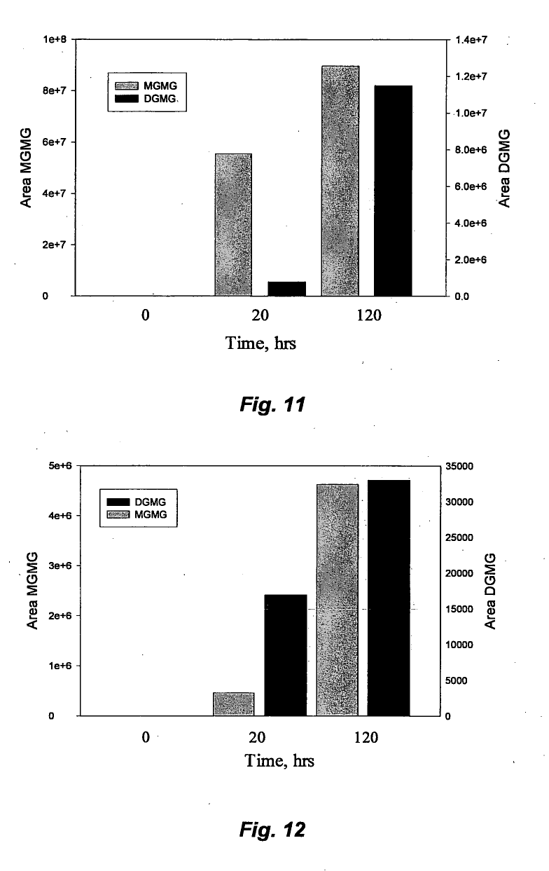

The samples were analyzed for glucoglycerolipids composition by LC-MS. The amounts of monoglucosyl-monoglyceride (MGMG) and diglucosyl-monoglyceride (DGMG) increased significantly over time (FIGS. 11 and 12).

From LC-MS analysis it can be concluded that the transglucosidase can transfer glucosyl moieties from maltodextrin and sucrose to monoglyceride.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Altschul, Stephen F., Gish, Warren, Miller, Webb, Myers, Eugene W., and Lipman, David J. (1990). Basic local alignment search tool. J. Mol. Biol. 215; 403-410.
Busquet, M.-P. et al, Appl Microbiol Biotechnol (1998) 50: 167-173.
Carter et al. J. Am. Chem. Soc. (1956), 78, 3735.
Carter et al. J. Lipid Res. (1961), 2(3), 215.
Carter et al. J. Lipid Res. (1961), 2(3), 223.
Coutinho, P. M. & Henrissat, B. (1999) “Carbohydrate-active enzymes: an integrated database approach” in “Recent Advances in Carbohydrate Bioengineering”, H. J. Gilbert, G. Davies, B. Henrissat and B. Svensson eds., The Royal Society of Chemistry, Cambridge, pp. 3-12.
Dartois et al. Biochemica Biophysica Acta, 1131: 253-360 (1993).
Dayhoff M O, Schwartz R M, Orcutt B C (1978). A model of evolutionary change in proteins. Atlas of protein sequence and structure Vol. 5, Suppl. 3; 345-352. National biomedical research foundation, Selver Spring, Md.
Gonnet G H, Cohen M A, Benner S A (1992). Exhaustive matching of the entire pretein sequence database. Science, 256; 1443.
Davies G, Henrissat B (1995) ‘Structures and mechanisms of glycosyl hydrolases’ Structure 3:853-859.
Hata, Y., Kitamoto, K., Gomi, K., Kumagai, C., Tamura, G., ‘Functional elements of the promoter region of the Aspergillus oryzae glaA gene encoding glucoamylase, Curr. Genet. 22 (2), 85-91 (1992).
Henikoff S and Henikoff J G (1992). Amino acid substitution matrices from protein blocks. Proc. Natl. Acad. Sci. USA, 89; 10915.
Henrissat B (1991) ‘A classification of glycosyl hydrolases based on amino-acid sequence similarities’. Biochem. J. 280:309-316.
Henrissat B, Bairoch A (1993) ‘New families in the classification of glycosyl hydrolases based on amino-acid sequence similarities’. Biochem. J. 293:781-788;
Henrissat B, Bairoch A (1996) ‘Updating the sequence-based classification of glycosyl hydrolases. Biochem. J. 316:695-696.
Henrissat B, Davies G J (1997) ‘Structural and sequence-based classification of glycoside hydrolases’. Curr. Op. Struct. Biol. 7:637-644.
Higgins, D. G., Bleasby, A. J. and Fuchs, R. (1992). CLUSTAL V: improved software for multiple sequence alignment. Computer Applications in the Biosciences (CABIOS), 8(2):189-191.
Higgins D G, Sharp P M (1988). A package for performing multiple sequence alignment on a microcomputer. Gene 73(1), 237-244.
Horwell D C, Trends Biotechnol. (1995) 13(4), 132-134.
Jones D T, Taylor W R, Thornton J M (1992). The rapid generation of mutation data matrices from protein sequences. Comut. Appl. Biosci. 8:3; 275.
Kato et al. Appl Environ Microbiol. 2002; 68(3): 1250-1256.
Larkin M. A., Blackshields G., Brown N. P., Chenna R., McGettigan P. A., McWilliam H.*, Valentin F.*, Wallace I. M., Wilm A., Lopez R.*, Thompson J. D., Gibson T. J. and Higgins D. G. (2007). ClustalW and ClustalX version 2. Bioinformatics 2007 23(21): 2947-2948.
Monsan, P. et al, Enzymatic production of a-butylglucoside and its fatty acid esters. Ann New York Acad Sci 799: 633-641.
Nakamura, A., Nishimura, I., Yokoyama, A., Lee, D. G., Hidaka, M., Masaki, H., Kimura, A., Chiba, S, and Uozumi, T., ‘Cloning and sequencing of an alpha-glucosidase gene from Aspergillus niger and its expression in A. nidulans’, J. Biotechnol. 53 (1), 75-84 (1997).
Pomeranz, Y. Glycolipid-protein interaction in breadmaking, Bakers Dig. (1971), 45, 26-31.
Pomeranz, Y. (1987) in Modern Cereal Science and Technology.
Simon R J et al., PNAS (1992) 89(20), 9367-9371.
Thompson J. D., Higgins D. G., Gibson T. J. (1994). “CLUSTAL W: improving the sensitivity of progressive multiple sequence alignment through sequence weighting, position-specific gap penalties and weight matrix choice.” Nucleic Acids Res. 22:4673-4680.

1. A method of glycosylating a lipid having a free hydroxyl group, the method comprising contacting said lipid with a source of monosaccharide moiety and a transglycosidase enzyme.

2. A method according to claim 1, further comprising isolating the glycosylated lipid from the reaction mixture.

3. A method according to claim 1, further comprising purifying the glycosylated lipid.

4. A method according to claim 1, wherein the transglycosidase enzyme is a transglucosidase enzyme.

5. A method according to claim 1, wherein the transglycosidase enzyme is classified in Enzyme Classification (E.C.) 3.2.1.20.

6. A method according to claim 1, wherein the transglycosidase enzyme is classified in Glycoside Hydrolase Family 31 of the Carbohydrate-Active Enzymes database.

7. A method according to claim 1, wherein the transglycosidase enzyme is of fungal origin or has at least 50%, preferably at least 55%, such as at least 60%, at least 65%, at least 70%, at least 75%, at least 80%, at least 85%, at least 90%, at least 95%, at least 96%, at least 97%, at least 98% or at least 99%, sequence identity with a transglycosidase enzyme of fungal origin.

8. A method according to claim 1, wherein the transglycosidase enzyme originates from an Aspergillus species or has at least 50%, preferably at least 55%, such as at least 60%, at least 65%, at least 70%, at least 75%, at least 80%, at least 85%, at least 90%, at least 95%, at least 96%, at least 97%, at least 98% or at least 99%, sequence identity with a transglycosidase enzyme originating from an Aspergillus species.

9. A method according to claim 8, wherein the Aspergillus species is selected from the group consisting of Aspergillus niger, Aspergillus awamori, Aspergillus terreus, Aspergillus oryzae, Aspergillus nidulans, Aspergullus fumigatus and Aspergillus clavatus.

10. A method according to claim 1, wherein the transglycosidase enzyme is Aspergillus niger transglucosidase (SEQ ID No 2 or SEQ ID No. 4) or has at least 50%, preferably at least 55%, such as at least 60%, at least 65%, at least 70%, at least 75%, at least 80%, at least 85%, at least 90%, at least 95%, at least 96%, at least 97%, at least 98% or at least 99%, sequence identity therewith.

11. A method according to claim 1, wherein the lipid is a mono- or polyglycerolipid.

12. A method according to claim 11, wherein the lipid is a monoglycerolipid.

13. A method according to claim 11, wherein the lipid is a diglycerolipid.

14. A method according to claim 11, wherein the lipid is a polyglycerolipid.

15. A method according to claim 12, wherein the lipid is selected from a monoglyceride or a diglyceride.

16. A method according to claim 15, wherein the monoglyceride has one saturated or unsaturated acyl chain having 4 to 40 carbon atoms.

17. A method according to claim 16, wherein the monoglyceride has one saturated or unsaturated acyl chain having 10 to 22 carbon atoms.

18. A method according to claim 15, wherein the diglyceride has two saturated or unsaturated acyl chains each independently having 4 to 40 carbon atoms.

19. A method according to claim 18, wherein the diglyceride has two saturated or unsaturated acyl chains each independently having 10 to 22 carbon atoms.

20. A method according to claim 1, wherein the monosaccharide moiety is a glucose moiety.

21. A method according to claim 1, wherein the source of monosaccharide moiety is a disaccharide.

22. A method according to claim 21, wherein the disaccharide is selected from maltose, isomaltose, isomaltulose, lactose, sucrose, cellobiose, nigerose, kojibiose, trehalose and trehalulose.

23. A method according to claim 22, wherein the disaccharide is sucrose.

24. A method according to claim 1, wherein the source of monosaccharide moiety is an oligosaccharide.

25. A method according to claim 24, wherein the oligosaccharide is selected from maltodextrin, maltotriose, maltotetraose, maltopentaose, maltohexaose, maltoheptaose, melezitose, cellotriose, cellotetraose, cellopentaose, cellohexaose and celloheptaose.

26. A method according to claim 1, wherein the source of monosaccharide moiety is a polysaccharide.

27. A method according to claim 26, wherein the polysaccharide is selected from starch, amylose, amylopectin, glycogen, cellulose or a derivative thereof, alginic acid or a salt or derivative thereof, polydextrose, dextran, pectin, pullulan, carrageenan, locust bean gum and guar gum.

28. A method of transfer of a monosaccharide moiety to a lipid, as defined in claim 1, by contacting the lipid with a source of monosaccharide moiety, as defined in claim 1, and a transglycosidase enzyme, as defined in claim 1.

29. A food product improving composition including: a transglycosidase enzyme; a source of monosaccharide moiety; and a lipid.

30. A food product improving composition according to claim 29 including: a transglycosidase enzyme, as defined in claim 1; a source of monosaccharide moiety, as defined in claim 1; and a lipid, as defined in claim 1.

31. A food product improving composition according to claim 29, wherein the food product is selected from a dough and a baked product prepared from dough.

32. A food product improving composition according to claim 29, wherein the source of monosaccharide moiety is sucrose.

33. A method of improving the emulsification properties of a food product, the method comprising contacting the food product or a food product intermediate with a source of monosaccharide moiety, a lipid, and a transglycosidase enzyme.

34. A method according to claim 33 of improving the emulsification properties of a food product, the method comprising contacting the food product or food product intermediate with a source of monosaccharide moiety, as defined in claim 1, a lipid, as defined in claim 1, and a transglycosidase enzyme, as defined in claim 1.

35. A method according to claim 34, wherein the food product is selected from dough and a baked product prepared from dough, the method further comprising, if necessary, subjecting the resulting dough to baking under suitable conditions.

36. A method for in situ production of a glycosylated lipid in a food product, the method comprising contacting the food product or food product intermediate with a source of monosaccharide moiety, a lipid, and a transglycosidase enzyme.

37. A method according to claim 36 for in situ production of a glycosylated lipid in a food product, the method comprising contacting the food product or food product intermediate with a source of monosaccharide moiety, as defined in claim 1, a lipid, as defined in claim 1, and a transglycosidase enzyme, as defined in claim 1.

38. A method according to claim 37, wherein the food product is a baked product.

39. A method according to claim 1, wherein the food product intermediate is a dough.

40. A method according to claim 1, wherein the source of monosaccharide moiety is sucrose.

41. A method according to claim 1, wherein the source of monosaccharide moiety is added in an amount of up to 30% by weight.

42. A method according to claim 41 wherein the source of monosaccharide moiety is added in an amount of 10% to 20% by weight, such as, e.g. 8% to 12% by weight.

43. A method according to claim 33, wherein the lipid is selected from a monoglyceride or a diglyceride.

44. A method according to claim 43, wherein the lipid is added in an amount of up to 10, such as, e.g. up to 3, up to 1.5, or up to 1% by weight.

45. A food product formed from the composition of claim 29.

46. A food product produced by the method claim 33.

47. Use of a transglycosidase enzyme, for the transfer of a monosaccharide moiety to a lipid.

48. Use according to claim 47 of a transglycosidase enzyme, as defined in claim 1, for the transfer of a monosaccharide moiety to a lipid, as defined in claim 1.

49. Use according to claim 47 wherein said transfer of a monosaccharide moiety to a lipid takes place in situ in a food product or food product intermediate.

50. Use according to claim 47 wherein said transfer of a monosaccharide moiety takes place in situ in a laundry composition.

51. Use of a transglycosidase enzyme, as defined in claim 1, for improving the emulsification properties of a food product, said food product including a lipid, as defined in claim 1, and a source of monosaccharide moiety, as defined in claim 1.


SEQUENCE LISTING (4 sequences)

SEQ ID NO: 1 - DNA, 2892 bp, Aspergillus niger
tcactcttatccaccactgccccttcgcagccgcagtttaccattcctgcttccgcagatgtcggtgcgcagctgattgccaacatcgatgatcctcaggctgccgacgcgcagtcggtttgtccgggctacaaggcttcaaaagtgcagcacaattcacgtggattcactgccagtcttcagctcgcgggcaggccatgtaacgtatacggcacagatgttgagtccttgacactgtctgtggagtaccaggattcggatcgactgaatattcagattctccccactcatgttgactccacaaacgcttcttggtactttctttcggaaaacctggtccccagacccaaggcttccctcaatgcatctgtatcccagagcgacctttttgtgtcatggtcaaatgagccgtcgttcaatttcaaggtgatccgaaaggctacaggcgacgcgcttttcagtacagaaggcactgtgctcgtatatgagaatcagttcatcgaatttgtgaccgcgctccctgaagaatataacttgtatggccttggggagcatatcacgcaattccgcctccagagaaatgctaatctgaccatatatccttcggatgatggaacacctattgaccaaaacctctacggccaacatcccttctatctggatacaagatattacaaaggagataggcagaatgggtcttatattcccgtcaaaagcagcgaggctgatgcctcgcaagattatatctccctctctcatggcgtgtttctgaggaactctcatggacttgagatactcctccggtctcaaaaattgatctggcggaccctaggtggaggaatcgatctcaccttctactcaggccccgccccggccgatgttaccaggcaatatcttaccagcactgtgggattaccggccatgcagcaatacaacactcttggattccaccaatgtcgttggggctacaacaactggtcggatctggcggacgttgttgcgaactttgagaagtttgagatcccgttggaatatatctggaccgatattgactacatgcacggatatcgcaactttgacaacgatcaacatcgcttttcctacagtgagggcgatgaatttctcagcaagctacatgagagtggacgctactatgtacccattgttgatgcggcgctctacattcctaatcccgaaaatgcctctgatgcatacgctacgtatgacagaggagctgcggacgacgtcttcctcaagaatcccgatggtagcctctatattggagccgtttggccaggatatacagtcttccccgattggcatcatcccaaggcagttgacttctgggctaacgagcttgttatctggtcgaagaaagtggcgttcgatggtgtgtggtacgacatgtctgaagtttcatccttctgtgtcgggagctgtggcacaggtaacctgactctgaacccggcacacccatcgtttcttctccccggtgagcctggtgatatcatatatgattacccagaggctttcaatatcaccaacgctacagaggcggcgtcagcttcggcgggagcttccagtcaggctgcagcaaccgcgaccaccacgtcgacttcggtatcatatctgcggacaacgcccacgcctggtgtccgcaatgttgagcacccaccctatgtgatcaaccatgaccaagaaggccatgatctcagtgtccatgcggtgtcgccgaatgcaacgcatgttgatggtgttgaggagtatgatgtgcacggtctctacggacatcaaggattgaacgctacctaccaaggtctgcttgaggtctggtctcataagcggcggccatttattattggccgctcaaccttcgctggctctggcaaatgggcaggccactggggcggcgacaactattccaaatggtggtccatgtactactccatctcgcaagccctctccttctcacttttcggcattccgatgtttggtgcggacacctgtgggtttaacggaaactccgatgaggagctctgcaaccgatggatgcaactgtccgcattcttcccattctaccgaaaccacaatgagctctccacaatcccacaggagccttatcggtgggcttctgttattgaagcaaccaagtccgccatgagaattcggtacgccatcctaccttacttttatacgttgtttgacctggcccacaccacgggctccactgtaatgcgcgcactttcctgggaattccctaatgacccaacattggctgcggttgagactcaattcatggttgggccggccatcatggtggtcccggtattggagcctctggtcaatacggtcaagggcgtattcccaggagttggacatggcgaagtgtggtacgattggtacacccaggctgcagttgatgcgaagcccggggtcaacacgaccatttcggcaccattgggccacatcccagtttatgtacgaggtggaaacatcttgccgatgcaagagccggcattgaccactcgtgaagcccggcaaaccccgtgggctttgctagctgcactaggaagcaatggaaccgcgtcggggcagctctatctcgatgatggagagagcatctaccccaatgccaccctccatgtggacttcacggcatcgcggtcaagcctgcgctcgtcggctcaaggaagatggaaagagaggaacccgcttgctaatgtgacggtgctcggagtgaacaaggagccctctgcggtgaccctgaatggacaggccgtatttcccgggtctgtcacgtacaattctacgtcccaggttctctttgttggggggctgcaaaacttgacgaagggcggcgcatgggcggaaaactgggtattggaatggtag

SEQ ID NO: 2 - protein, 963 aa, Aspergillus niger
SLLSTTAPSQPQFTIPASADVGAQLIANIDDPQAADAQSVCPGYKASKVQHNSRGFTASLQLAGRPCNVYGTDVESLTLSVEYQDSDRLNIQILPTHVDSTNASWYFLSENLVPRPKASLNASVSQSDLFVSWSNEPSFNFKVIRKATGDALFSTEGTVLVYENQFIEFVTALPEEYNLYGLGEHITQFRLQRNANLTIYPSDDGTPIDQNLYGQHPFYLDTRYYKGDRQNGSYIPVKSSEADASQDYISLSHGVFLRNSHGLEILLRSQKLIWRTLGGGIDLTFYSGPAPADVTRQYLTSTVGLPAMQQYNTLGFHQCRWGYNNWSDLADVVANFEKFEIPLEYIWTDIDYMHGYRNFDNDQHRFSYSEGDEFLSKLHESGRYYVPIVDAALYIPNPENASDAYATYDRGAADDVFLKNPDGSLYIGAVWPGYTVFPDWHHPKAVDFWANELVIWSKKVAFDGVWYDMSEVSSFCVGSCGTGNLTLNPAHPSFLLPGEPGDIIYDYPEAFNITNATEAASASAGASSQAAATATTTSTSVSYLRTTPTPGVRNVEHPPYVINHDQEGHDLSVHAVSPNATHVDGVEEYDVHGLYGHQGLNATYQGLLEVWSHKRRPFIIGRSTFAGSGKWAGHWGGDNYSKWWSMYYSISQALSFSLFGIPMFGADTCGFNGNSDEELCNRWMQLSAFFPFYRNHNELSTIPQEPYRWASVIEATKSAMRIRYAILPYFYTLFDLAHTTGSTVMRALSWEFPNDPTLAAVETQFMVGPAIMVVPVLEPLVNTVKGVFPGVGHGEVWYDWYTQAAVDAKPGVNTTISAPLGHIPVYVRGGNILPMQEPALTTREARQTPWALLAALGSNGTASGQLYLDDGESIYPNATLHVDFTASRSSLRSSAQGRWKERNPLANVTVLGVNKEPSAVTLNGQAVFPGSVTYNSTSQVLFVGGLQNLTKGGAWAENWVLEW

SEQ ID NO: 3 - DNA, 4965 bp, Aspergillus niger
gcatgccatgagttgttgggccggcctgaaggatccatcattgggaccaagggcatcatccatgcgctacggagtactttcggagaatcagcacccctgcacaaagcattgtcaatgtgttttcttatgtcaaaagctgacagagtctgaggctcgctgacgatgggattcatgctaatgacggtccgaaagagctttcacgtaacactggtgaacatcccactcgggaagccgagacttgtgacctacttagtcaaatgagatgattatcaaagccattaaatgcctcgctgtcaggggccctggtaagtgtcttcattaatcgaaacccatcttcattcgtccccgccttcagtgctcatcattttaggtttagaagcaagattgagtgccacctgctttacaaaccagcatgggtagtctgctgttgaaattcttcaccgggagcattctggggaaggtgcaaaaggcggcgcgaagtggtcgggtcgcgattgtagtctggattggagcacaagaatcgtcagagccgaagcccgaactgagggggtctcggtcatttatcgggatgagagccaatcagcgtgcgctcatcatctgatcgtctggctgccaggcccctcaggcatcaatacggtactcggcagtatccactcccgtttctccggtgcaacaaatcatcgttggagaatccccagctcccccgccaactggggtcgatgcttctccagttgtcctggtttctcccatgaactcgcttacgataagctgctgtaccagcccaccagcacaacaatatcttcaatcaggtaggtgcttgttcgttacctgccccatcctctcctcttcttcggtcattatgaactcaattcggtcgctagctttgccgattctccgcagtccataaaaatatatctgcatttgccccttacacgtcgggaattcaccggcgcaatgagccttcgggtatggtcgcacagcgtcatgtcaataggaggctgctcctagtggtgatctactagtcgcctcaacacagcaatatataaataacaagagcattccttgagcacatctgggtaatagctgttccattctcatcaaggattacgcgaccgtgcctcgagcctccttaagcgagccatggtgaagttgacgcatctccttgccagagcatggcttgtccctctggcttatggagcgagccagtcactcttatccaccactgccccttcgcagccgcagtttaccattcctgcttccgcagatgtcggtgcgcagctgattgccaacatcgatgatcctcaggctgccgacgcgcagtcggtttgtccgggctacaaggcttcaaaagtgcagcacaattcacgtggattcactgccagtcttcagctcgcgggcaggccatgtaacgtatacggcacagatgttgagtccttgacactgtctgtggagtaccaggattcggatcgactgaatattcagattctccccactcatgttgactccacaaacgcttcttggtactttctttcggaaaacctggtccccagacccaaggcttccctcaatgcatctgtatcccagagcgacctttttgtgtcatggtcaaatgagccgtcgttcaatttcaaggtgatccgaaaggctacaggcgacgcgcttttcagtacagaaggcactgtgctcgtatatgagaatcagttcatcgaatttgtgaccgcgctccctgaagaatataacttgtatggccttggggagcatatcacgcaattccgcctccagagaaatgctaatctgaccatatatccttcggatgatggaacacctattgaccagtgagtactgatatcccgcccgtatcttctggttctactcttgaaacttactcgtcctagaaacctctacggccaacatcccttctatctggatacaagatattacaaaggagataggcagaatgggtcttatattcccgtcaaaagcagcgaggctgatgcctcgcaagattatatctccctctctcatggcgtgtttctgaggaactctcatggacttgagatactcctccggtctcaaaaattgatctggcggaccctaggtggaggaatcgatctcaccttctactcaggccccgccccggccgatgttaccaggcaatatcttaccagcactgtgggattaccggccatgcagcaatacaacactcttggattccaccaatgtcgttggggctacaacaactggtcggatctggcggacgttgttgcgaactttgagaagtttgagatcccgttggaatatatctggtgcgtattgtactggtttatggtatctcaaaacagtctaacaggcacttaggaccgatattgactacatgcacggatatcgcaactttgacaacgatcaacatcgcttttcctacagtgagggcgatgaatttctcagcaagctacatgagagtggacgctactatgtacccattgttgatgcggcgctctacattcctaatcccgaaaatgcctctgatgcgtaagtgtctagtgacaaattatattactgcctgtatgctaattagcgatacagatacgctacgtatgacagaggagctgcggacgacgtcttcctcaagaatcccgatggtagcctctatattggagccgtttggccaggatatacagtcttccccgattggcatcatcccaaggcagttgacttctgggctaacgagcttgttatctggtcgaagaaagtggcgttcgatggtgtgtggtacgacatgtctgaagtttcatccttctgtgtcgggagctgtggcacaggtaacctgactctgaacccggcacacccatcgtttcttctccccggtgagcctggtgatatcatatatgattacccagaggctttcaatatcaccaacgctacagaggcggcgtcagcttcggcgggagcttccagtcaggctgcagcaaccgcgaccaccacgtcgacttcggtatcatatctgcggacaacgcccacgcctggtgtccgcaatgttgagcacccaccctatgtgatcaaccatgaccaagaaggccatgatctcagtgtccatgcggtgtcgccgaatgcaacgcatgttgatggtgttgaggagtatgatgtgcacggtctctacggacatcaaggattgaacgctacctaccaaggtctgcttgaggtctggtctcataagcggcggccatttattattggccgctcaaccttcgctggctctggcaaatgggcaggccactggggcggcgacaactattccaaatggtggtccatgtactactccatctcgcaagccctctccttctcacttttcggcattccgatgtttggtgcggacacctgtgggtttaacggaaactccgatgaggagctctgcaaccgatggatgcaactgtccgcattcttcccattctaccgaaaccacaatgagctctccacaatcccacaggagccttatcggtgggcttctgttattgaagcaaccaagtccgccatgagaattcggtacgccatcctaccttacttttatacgttgtttgacctggcccacaccacgggctccactgtaatgcgcgcactttcctgggaattccctaatgacccaacattggctgcggttgagactcaattcatggttgggccggccatcatggtggtcccggtattggagcctctggtcaatacggtcaagggcgtattcccaggagttggacatggcgaagtgtggtacgattggtacacccaggctgcagttgatgcgaagcccggggtcaacacgaccatttcggcaccattgggccacatcccagtttatgtacgaggtggaaacatcttgccgatgcaagagccggcattgaccactcgtgaagcccggcaaaccccgtgggctttgctagctgcactaggaagcaatggaaccgcgtcggggcagctctatctcgatgatggagagagcatctaccccaatgccaccctccatgtggacttcacggcatcgcggtcaagcctgcgctcgtcggctcaaggaagatggaaagagaggaacccgcttgctaatgtgacggtgctcggagtgaacaaggagccctctgcggtgaccctgaatggacaggccgtatttcccgggtctgtcacgtacaattctacgtcccaggttctctttgttggggggctgcaaaacttgacgaagggcggcgcatgggcggaaaactgggtattggaatggtagtgtcagccacaagccaggtgtgcgcgtacagcatgcaacatgggaacgatgctctgcaatgtagctctttggttataattcaaaattcaacttccacctttgtttcaccggcggccacggcattcctgcatgactaacgttctgtaaatggacccgataacacccagcacgttgcagcagagaaggtactctctcacacgcactgctctttatagttgccgagacggccgccgaggagaaaaccgccggcctgtggccactattcgctggaaggaaccctgccagtcgaacacacccgcccgtgatcgccaggggccgatggatttccccccgcatccttgtcggttcatgagtgaagactttaaatcccatctagctgacggtcgggtacatcaataactggcagcctagtttccaagacacggagaagcatgtaatcgctatttatagaatgctgggatcggacccgtcgaatggtcttccgatgggaagtgacaactcacattgtcatgttggccttactcaatccaacgggatctgacctgctttggctaacctagtataaatcagcatgtctctcctttgatacatcggatcgttcctcaaatatagttatatcttcgaaaaattgacaagaaggatgacaatctttctgtttctggccattttcgtggctacagctctggcagccacgcctgcag

SEQ ID NO: 4 - protein, 985 aa, Aspergillus niger
MVKLTHLLARAWLVPLAYGASQSLLSTTAPSQPQFTIPASADVGAQLIANIDDPQAADAQSVCPGYKASKVQHNSRGFTASLQLAGRPCNVYGTDVESLTLSVEYQDSDRLNIQILPTHVDSTNASWYFLSENLVPRPKASLNASVSQSDLFVSWSNEPSFNFKVIRKATGDALFSTEGTVLVYENQFIEFVTALPEEYNLYGLGEHITQFRLQRNANLTIYPSDDGTPIDQNLYGQHPFYLDTRYYKGDRQNGSYIPVKSSEADASQDYISLSHGVFLRNSHGLEILLRSQKLIWRTLGGGIDLTFYSGPAPADVTRQYLTSTVGLPAMQQYNTLGFHQCRWGYNNWSDLADVVANFEKFEIPLEYIWTDIDYMHGYRNFDNDQHRFSYSEGDEFLSKLHESGRYYVPIVDAALYIPNPENASDAYATYDRGAADDVFLKNPDGSLYIGAVWPGYTVFPDWHHPKAVDFWANELVIWSKKVAFDGVWYDMSEVSSFCVGSCGTGNLTLNPAHPSFLLPGEPGDIIYDYPEAFNITNATEAASASAGASSQAAATATTTSTSVSYLRTTPTPGVRNVEHPPYVINHDQEGHDLSVHAVSPNATHVDGVEEYDVHGLYGHQGLNATYQGLLEVWSHKRRPFIIGRSTFAGSGKWAGHWGGDNYSKWWSMYYSISQALSFSLFGIPMFGADTCGFNGNSDEELCNRWMQLSAFFPFYRNHNELSTIPQEPYRWASVIEATKSAMRIRYAILPYFYTLFDLAHTTGSTVMRALSWEFPNDPTLAAVETQFMVGPAIMVVPVLEPLVNTVKGVFPGVGHGEVWYDWYTQAAVDAKPGVNTTISAPLGHIPVYVRGGNILPMQEPALTTREARQTPWALLAALGSNGTASGQLYLDDGESIYPNATLHVDFTASRSSLRSSAQGRWKERNPLANVTVLGVNKEPSAVTLNGQAVFPGSVTYNSTSQVLFVGGLQNLTKGGAWAENWVLEW